(19) United States
(12) Patent Application Publication
(10) Pub. No.: US 2010/0152595 A1
(43) Pub. Date: Jun. 17, 2010

(54) AUTOMATED NOISE REDUCTION SYSTEM FOR PREDICTING ARRHYTHMIC DEATHS

(75) Inventors: James E. Skinner, Bangor, PA (US); Jerry M. Anchin, Boca Raton, FL (US); David H. Fater, Delray Beach, FL (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Non-Linear Medicine, Inc., Boca raton, FL (US)

(21) Appl. No.: 12/439,637

(22) PCT Filed: Aug. 30, 2007
(86) PCT No.: PCT/US07/77175

§ 371 Date: Mar. 5, 2010

Related U.S. Application Data
(60) Provisional application No. 60/824,170, filed on Aug. 31, 2006.

(51) Int. Cl. A61B 5/0402; A61B 5/0476

(52) U.S. Cl. 600/509; 600/544

(57) ABSTRACT

Provided are methods, systems, and computer readable media for reducing noise associated with electrophysiological data for more effectively predicting an arrhythmic death.

### CROSS REFERENCE TO RELATED PATENT APPLICATIONS

This application claims priority to U.S. Provisional Application No. 60/824,170 filed Aug. 31, 2006, herein incorporated by reference in its entirety.

### BACKGROUND

The present methods, systems, and computer readable media are directed toward evaluating electrophysiological data. Electrophysiological data can include, but is not limited to, electrocardiogram (ECG/EKG) data, electroencephalogram (EEG) data, and the like. More particularly, the present methods, systems, and computer readable media are directed to an automated system and method for evaluating electrophysiological data for detecting and/or predicting arrhythmic death.

Analysis of R-R intervals (RRi) observed in the electrocardiogram or of spikes seen in the electroencephalogram can predict future clinical outcomes, such as sudden cardiac death or epileptic seizures. An R-R interval is a time duration between two consecutive R waves of an ECG or an EEG. An R-R interval can be, for example, in the range of 0.0001 seconds to 5 seconds. Such analyses and predictions are statistically significant when used to discriminate outcomes between large groups of patients who either do or do not manifest the predicted outcome.

Such analyses and predictions suffered inaccuracy problems due to analytic measures (1) being stochastic (i.e., based on random variation in the data), (2) requiring stationarity (i.e., the system generating the data cannot change during the recording), and (3) being linear (i.e., insensitive to nonlinearities in the data which are referred to in the art as “chaos”).

Many techniques were developed to address these issues, including “D2”, “D2i”, and “PD2”. D2 enables the estimation of the dimension of a system or its number of degrees of freedom from an evaluation of a sample of data generated. Several investigators have used D2 on biological data. However, it has been shown that the presumption of data stationarity cannot be met.

Another theoretical description, the Pointwise Scaling Dimension or “D2i”, was developed that is less sensitive to the non-stationarities inherent in data from the brain, heart or skeletal muscle. This is perhaps a more useful estimate of dimension for biological data than the D2. However, D2i still has considerable errors of estimation that might be related to data non-stationarities.

A Point Correlation Dimension algorithm (PD2) was been developed that can detect changes in dimension in non-stationary data (i.e., data made by linking subepochs from different chaotic generators).

To address the failings of these various techniques, an improved PD2 algorithm, labeled the “PD2i” to emphasize its time-dependency, was developed. The PD2i, also referred to herein as a data processing routine, uses an analytic measure that is deterministic and based on caused variation in the data. The algorithm does not require data stationarity and actually tracks non-stationary changes in the data. Also, the PD2i is sensitive to chaotic as well as non-chaotic, linear data. The PD2i is based on previous analytic measures that are, collectively, the algorithms for estimating the correlation dimension, but it is insensitive to data non-stationarities. Because of this feature, the PD2i can predict clinical outcomes with high sensitivity and specificity that the other measures cannot. The PD2i algorithm is described in detail in U.S. Pat. Nos. 5,709,214 and 5,720,294, hereby incorporated by reference.

For analysis by the PD2i, an electrophysiological signal is amplified (gain of 1,000) and digitized (1,000 Hz). The digitized signal may be further reduced (e.g. conversion of ECG data to RR-interval data) prior to processing. Analysis of RR-interval data has been repeatedly found to enable risk-prediction between large groups of subjects with different pathological outcomes (e.g. ventricular fibrillation “VF”, ventricular tachycardia “VT”, or arrhythmic death “AD”). It has been shown that, using sampled RR data from high risk patients, PD2i could discriminate those that later went into VF from those that did not.

For RR-interval data made from a digital ECG that is acquired with the best low-noise preamps and fast 1,000-Hz digitizers, there is still a low-level of noise that can cause problems for nonlinear algorithms. The algorithm used to make the RR-intervals can also lead to increased noise. The most accurate of all RR-interval detectors uses a 3-point running “convexity operator.” For example, 3 points in a running window that goes through the entire data can be adjusted to maximize its output when it exactly straddles an R-wave peak; point 1 is on the pre R-wave baseline, point 2 is atop the R-wave, point 3 is again on the baseline. The location of point 2 in the data stream correctly identifies each R-wave peak as the window goes through the data. This algorithm will produce considerably more noise-free RR data than an algorithm which measures the point in time when an R-wave goes above a certain level or is detected when the dV/dt of each R-wave is maximum.

The best algorithmically calculated RR-intervals still will have a low-level of noise that is observed to be approximately +/−5 integers, peak-to-peak. This 10 integer range is out of 1000 integers for an average R-wave peak (i.e., 1% noise). With poor electrode preparation, strong ambient electromagnetic fields, the use of moderately noisy preamps, or the use of lower digitizing rates, the low-level noise can easily increase. For example, at a gain where 1 integer=1 msec (i.e., a gain of 25% of a full-scale 12-bit digitizer), this best noise level of 1% can easily double or triple, if the user is not careful with the data acquisition. This increase in noise often happens in a busy clinical setting, and thus post-acquisition consideration of the noise level must be made.

To address this issue of noise, a noise consideration algorithm (NCA), was developed. The NCA is more fully described in U.S. patent application Ser. No. 10/353,849, hereby incorporated by reference.

Even with the improvements in R-R interval analysis brought about by the PD2i data processing routine and the NCA, there still exists a need for automated methods, systems, and computer readable media for improving noise reduction and prediction of biological outcome determined by PD2i calculation.

### SUMMARY

Provided are automated methods, systems, and computer readable media for reducing noise associated with electrophysiological data for more effectively predicting an arrhythmic death.

Additional advantages will be set forth in part in the description which follows or may be learned by practice of the methods, systems, and computer readable media. The advantages will be realized and attained by means of the elements and combinations particularly pointed out in the appended claims. It is to be understood that both the foregoing general description and the following detailed description are exemplary and explanatory only and are not restrictive of the methods, systems, and computer readable media, as claimed.

### BRIEF DESCRIPTION OF THE DRAWINGS

The accompanying drawings, which are incorporated in and constitute a part of this specification, illustrate embodiments and together with the description, serve to explain the principles of the methods, systems, and computer readable media:

FIG. 1 is an exemplary operating environment;

FIG. 2 is an exemplary method flow diagram;

FIG. 3 is an exemplary EEG method flow diagram;

FIG. 4 is an exemplary PD2i data processing routine method flow diagram;

FIG. 5 is an exemplary outlier removal method flow diagram;

FIG. 6 A-B is an exemplary NCA method flow diagram;

FIG. 7 is an exemplary TZA method flow diagram;

FIG. 8 A-B illustrates an exemplary method flow diagram;

FIG. 9 illustrates R-waves digitized at 100 Hz vs those digitized at 1000 Hz.

FIG. 10 shows that different ways of detecting the R-R intervals have important implications for noise content in the data;

FIG. 11 shows an example of data that are, by definition, non-stationary;

FIG. 12 shows that removing a bit (i.e., dividing the amplitude by half) does not significantly alter the mean or distribution of a nonlinear measure;

FIG. 13 shows that the three lobes of the heartbeat attractor projected on to two dimensions in phase space are seemingly quite large, just as they are in the Lorenz and Sine-wave attractors;

FIG. 14 shows the effect of removing a noise bit on a nonlinear measure of a low-noise heartbeat file;

FIG. 15 shows a similar effect to that seen in FIG. 14, but uses Lorenz data and a time plot of the results instead of a histogram;

FIG. 16 shows an example of multiple PD2i scores in the transition zone between 1.4 and 1.6, when the apriori TZA threshold has been set a 1.40;

FIG. 17 shows RR and PD2i data from 18 patients who died of defined sudden arrhythmic (AD) within the 1-year of follow-up and 18 controls, each of whom had a documented acute myocardial infarction (AMI) and lived for at least the 1-year of follow-up;

FIG. 18 shows nonlinear results (PD2i) when the physiological data contain artifacts (arrhythmias, movement artifacts);

FIG. 19 illustrates the same data file and results as in the FIG. 18, but the artifacts have been removed by a linear spline that overwrites them;

FIG. 20 shows that the nonlinear PD2i detects changes in the degrees of freedom (dimensions) in data that have sub-epochs with similar means and standard deviations;

FIG. 21 shows electroencephalographic data (EEG) from a sleeping cat thought to be generating steady-state sleep data;

FIG. 22 shows the PD2i distributions for data and for its randomized-phase surrogate;

FIG. 23 shows that the PD2i-distributions are essentially the same and that increasing data length results in the PD2i's of the larger distributions becoming more unit-normal in appearance;

FIG. 24 illustrates the effects of adding noise to Lorenz Data (LOR) on its relative separation from it randomized-phase surrogate;

FIG. 25 A-D illustrates: A. the PD2i Algorithm and its comparison to the other time-dependent algorithm for calculating degrees of freedom, the Pointwise D2 (D2i). B. The effect on PD2i of adding ±5 integers of noise to the data. C. The PD2i of the randomized phase surrogate of the data. D. The power spectrum of the data and its surrogate (identical). E. The effect on PD2i of adding ±14 integers of noise to the data;

FIG. 26 shows a plot of % N of accepted PD2i vs noise content of Lorenz data;

FIG. 27 shows the same effect as in FIG. 26, but with the noise content (LOR+% noise) and % N shown for the PD2i distributions;

FIG. 28 shows the use of PD2i of heartbeats in defining dementia (Alzheimer's Disease) and cases of syncope;

FIG. 29 A-C shows how PD2i is calculated from vectors made from two samples of data points;

FIG. 30 shows how the Correlation Integral, made from vector difference lengths according to the mathematical model for PD2i (in the limit as Ni approaches infinity) appears for large data lengths and more realistic ones of finite data length;

FIG. 31 shows two ways to determine Tau, the number of data points skipped over to select those to be used in the ij-vector pairs as coordinates for making VDL's;

FIG. 32 shows that both a “Bad Heart” and a “Bad Brain” are required to cause the dynamical instability of ventricular fibrillation (VF);

FIG. 33 shows a nonlinear analysis of the PD2i of the R-R intervals of an AD patient who showed two large PVCs (upper, arrows) one of which led to ventricular fibrillation (see FIGS. 35 and 36) and the other did not;

FIG. 34 shows that the R-R intervals of the above AD patient are not really flat, but have a sinusoidal oscillation with a period of 6 to 8 heartbeats;

FIG. 35 shows that the ECG of the above AD patient in which a PVC (large downward deflection) occurs just after the peak of the last T-wave and initiates a small rapid rotor that then leads to a slower larger one; and

FIG. 36 shows the coupling interval of the PVC that does not evoke a rotor (PVC No R-wave) and the one that does are precisely the same, as the downward deflections of both traces beginning at the far left overlap completely up to the T-wave peak.

### DETAILED DESCRIPTION

Before the present methods, systems, and computer readable media are disclosed and described, it is to be understood that the methods, systems, and computer readable media are not limited to specific synthetic methods, specific components, or to particular compositions, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

### I. Systems

Provided is an automated system for reducing noise associated with electrophysiological data, such as data from an ECG/EKG, an EEG and the like, used in predicting a biological outcome, such as arrhythmic death. The system can comprise a processor coupled to receive the electrophysiological data and a storage device with noise correction software in communication with the processor, wherein the noise correction software controls the operation of the processor and causes the processor to execute any functions of the methods provided herein for reducing noise associated with electrophysiological data used in predicting an arrhythmic death.

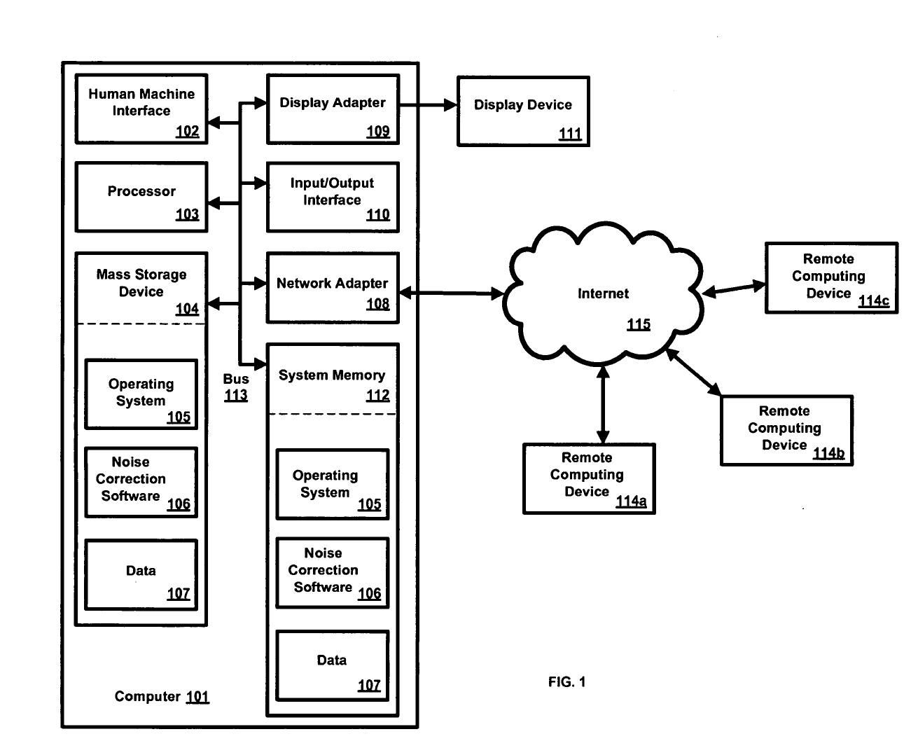

One skilled in the art will appreciate that this is a functional description and that respective functions can be performed by software, hardware, or a combination of software and hardware. A function can be software, hardware, or a combination of software and hardware. The functions can comprise the Noise Correction Software 106 as illustrated in FIG. 1 and described herein. In one exemplary aspect, the functions can comprise a computer 101 as illustrated in FIG. 1 and described herein.

FIG. 1 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The systems and methods can be operational with numerous other general purpose or special purpose computing system environments or configurations. Examples of well known computing systems, environments, and/or configurations that can be suitable for use with the system and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

In another aspect, the processing of the disclosed systems and methods can be performed by software components. The systems and methods can be described in the general context of computer instructions, such as program modules, being executed by a computer. Generally, program modules comprise routines, programs, objects, components, data structures, etc. that perform particular tasks or implement particular abstract data types. The system and methods can also be practiced in distributed computing environments where tasks are performed by remote processing devices that are linked through a communications network. In a distributed computing environment, program modules can be located in both local and remote computer storage media including memory storage devices.

Further, one skilled in the art will appreciate that the system and methods disclosed herein can be implemented via a general-purpose computing device in the form of a computer 101. The components of the computer 101 can comprise, but are not limited to, one or more processors or processing units 103, a system memory 112, and a system bus 113 that couples various system components including the processor 103 to the system memory 112.

The system bus 113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 113, and all buses specified in this description can also be implemented over a wired or wireless network connection and each of the subsystems, including the processor 103, a mass storage device 104, an operating system 105, Noise Correction software 106, data 107, a network adapter 108, system memory 112, an Input/Output Interface 110, a display adapter 109, a display device 111, and a human machine interface 102, can be contained within one or more remote computing devices 114a,b,c at physically separate locations, connected through buses of this form, in effect implementing a fully distributed system.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM). The system memory 112 typically contains data such as data 107 and/or program modules such as operating system 105 and Noise Correction software 106 that are immediately accessible to and/or are presently operated on by the processing unit 103.

In another aspect, the computer 101 can also comprise other removable/non-removable, volatile/non-volatile computer storage media. By way of example, FIG. 1 illustrates a mass storage device 104 which can provide non-volatile storage of computer code, computer readable instructions, data structures, program modules, and other data for the computer 101. For example and not meant to be limiting, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and Noise Correction software 106. Each of the operating system 105 and Noise Correction software 106 (or some combination thereof) can comprise elements of the programming and the Noise Correction software 106. Data 107 can also be stored on the mass storage device 104. Data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 101 via an input device (not shown). Examples of such input devices comprise, but are not limited to, a keyboard, pointing device (e.g., a “mouse”), a microphone, a joystick, a scanner, and the like. These and other input devices can be connected to the processing unit 103 via a human machine interface 102 that is coupled to the system bus 113, but can be connected by other interface and bus structures, such as a parallel port, game port, an IEEE 1394 Port (also known as a Firewire port), a serial port, or a universal serial bus (USB).

In yet another aspect, a display device 111 can also be connected to the system bus 113 via an interface, such as a display adapter 109. It is contemplated that the computer 101 can have more than one display adapter 109 and the computer 101 can have more than one display device 111. For example, a display device can be a monitor, an LCD (Liquid Crystal Display), or a projector. In addition to the display device 111, other output peripheral devices can comprise components such as speakers (not shown) and a printer (not shown) which can be connected to the computer 101 via Input/Output Interface 110.

The computer 101 can operate in a networked environment using logical connections to one or more remote computing devices 114a,b,c. By way of example, a remote computing device can be a personal computer, portable computer, a server, a router, a network computer, a peer device or other common network node, and so on. Logical connections between the computer 101 and a remote computing device 114a,b,c can be made via a local area network (LAN) and a general wide area network (WAN). Such network connections can be through a network adapter 108. A network adapter 108 can be implemented in both wired and wireless environments. Such networking environments are conventional and commonplace in offices, enterprise-wide computer networks, intranets, and the Internet 115.

For purposes of illustration, application programs and other executable program components such as the operating system 105 are illustrated herein as discrete blocks, although it is recognized that such programs and components reside at various times in different storage components of the computing device 101, and are executed by the data processor(s) of the computer. An implementation of Noise Correction software 106 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods- or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods, systems, and computer readable media can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

### II. Methods

A. Electrophysiological Data Considerations

There are several considerations that should be taken into account for the data input (i.e. R-R interval data) into the automated methods, systems, and computer readable media provided. These considerations include noise considerations, non-stationarity considerations, and data length considerations.

i. Noise Considerations

There are various noise considerations to account for in electrophysiological data subjected to automated nonlinear analysis. Two such sources include inherent amplifier noise and inherent descretization errors (digitization rate).

Electrophysiological data is usually amplified, and the amplifier noise, typically about 5 uV, is also amplified. For 12-bit digitizers at full scale (4112 integers, rounded off to 4000), the amplifier gain is set so that 25% of full scale (i.e., 1000 integers) is 1 uV=1 integer. That is, the usual amplitude of an R-wave, which is around 1000 uV, is equal to 1000 integers. Therefore the inherent noise of 5 uV is equal to 5 integers. This inherent noise in the R-wave amplitude (amplitude domain) is translated directly into the time-domain as well (e.g., during R-R interval detection).

In the detection of the R-wave peak, where it is defined in one of the time-bins of a digitizer (e.g., for a digitization rate of 1000 Hz, one bin=1 msec) translates directly into to the uncertainty of two R-waves required to determine the time interval between them. That is, for a digitization rate of 100 Hz the descretization error is 2 divided by 100 which equals 2% error in the time domain. For a digitization rate of 1000 Hz this is reduced to 2 divided by 1000 or 0.2% descretization error. This error is additive (root mean square) to that of the amplifier noise.

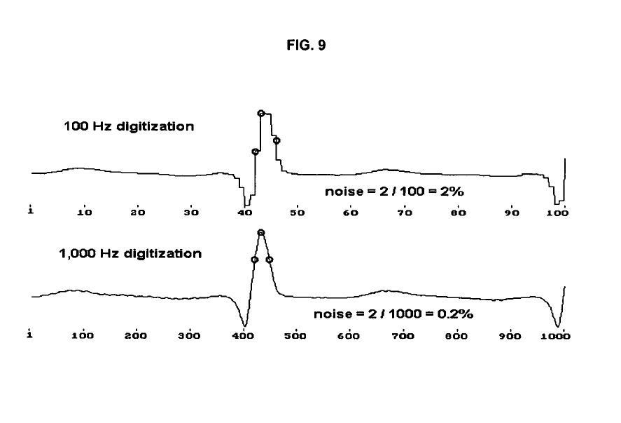

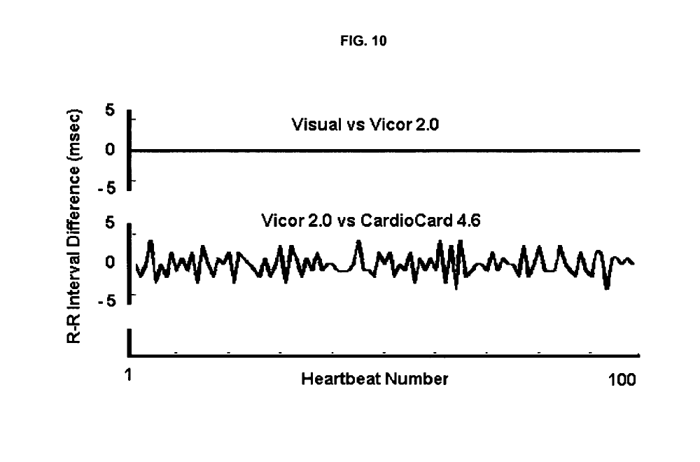

R-R interval data used for input into the methods, systems, and computer readable media provided can be obtained from various sources including an R-R Interval Detector. Like the amplifier above, the method of R-R Interval detection used can attribute to noise in the R-R interval data obtained. FIG. 9 Illustrates the difficulty a 3-point, running-window, peak-detector has in finding the peak of an R-wave digitized at 100 Hz vs that for the same R-wave digitized at 1000 Hz. Because of the large descretization error of ECGs digitized around 100 Hz (i.e., 2%), it is not possible to perform nonlinear analyses on them. Digitization rates around 250 Hz are also problematic in this regard. Table 1 shows that only 4 of 21 ECGs digitized at 256 Hz had nonlinear values that were significantly different from their filtered-noise (Randomized-Phase) surrogate. These significant four were for the files that had the lower mean values of the nonlinear measure (PD2i) and therefore required fewer data points. At 1000-Hz digitization rate, with all other features being the same, 100% of the files would have their nonlinear results be significantly different from their filtered-noise surrogates.

TABLE 1
Only 20% of files digitized at 256 Hz have nonlinear values (mean PD2i
of heartbeats) that are significantly different from their filtered-noise
surrogate (i.e., randomized-phase inverse- Fourier transform).

| Nonlinear | | Surrogate |
|---|---|---|
| Measure | SD | Measure |
| 2.81 | 0.56 | ns |
| 4.75 | 1.01 | ns |
| 1.75 | 0.41 | p ≦ 0.01 |
| 3.53 | 1.3 | ns |
| 4.37 | 1.52 | ns |
| 3.88 | 0.69 | ns |
| 4.18 | 0.8 | ns |
| 5.42 | 1.42 | rej |
| 4.78 | 1.06 | ns |
| 4.46 | 1.34 | ns |
| 3.85 | 1.26 | ns |
| 4.41 | 1.03 | ns |
| 1.8 | 0.72 | p ≦ 0.01 |
| 3.67 | 0.81 | ns |
| 3.84 | 0.88 | ns |
| 2.26 | 0.66 | ns |
| 1.72 | 0.95 | p ≦ 0.01 |
| 3.56 | 1.25 | ns |
| 2.77 | 0.85 | ns |
| 3.95 | 1.13 | ns |
| 1.39 | 0.9 | p ≦ 0.01 |

ii. Data Non-Stationarity Considerations

Another important consideration in data quality for nonlinear analysis is whether or not the data are stationary. Algorithms based on a Linear Stochastic model (e.g., standard deviation of normal to normal heartbeats, SDNN, power spectrum of the heartbeats, etc.) require data stationarity, as do many nonlinear algorithms. However, most electrophysiological data under the control of the nervous system, including the heartbeats, are quite non-stationary over time. This non-stationarity can be caused as electrophysiological data is acquired from a subject by, for example, the subject sneezing, suddenly moving, and the like. This is an example of a physiological non-stationarity, as the ergodotic properties of the heartbeat population will change (i.e., its mean, standard deviation, degrees of freedom, etc.).

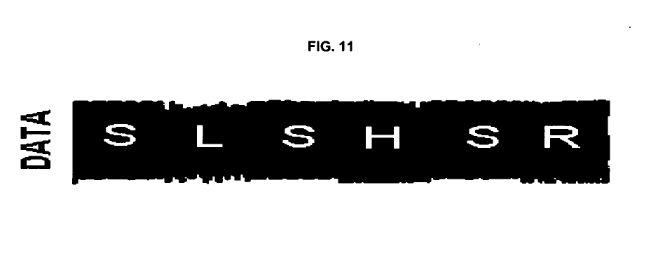

FIG. 11 shows an example of data that are, by definition, non-stationary. The nonstationary data (7,200 data-points) were created by linking sub-epochs made by different generators. The sub-epoch mean and SDs, are about the same, but the degrees of freedom are subtly different: sine wave (S, df=1.00); Lorenz (L, df=2.06); Henon (H, df=1.46) and random (R, df=infinity). These test data will be discussed several times herein. The overall epoch can be made by linking together sub-epochs of continuous outputs from an electronic sine-wave generator (S, continuous data), a Lorenz generator (L, continuous data), a Henon generator (H, map-function), and a random white-noise generator (R, continuous data). Each sub-epoch (1,200 data points each) can be linked together to make a 7,200 data-point non-stationary file with its amplitude being equivalent to the smaller R-waves of a cardiac patient (350 integers=0.35 mV). Each sub-epoch generator can have about the same dynamic range of amplitude and approximately the same mean, and standard deviation, but it does not have the same number of degrees of freedom. Many of the nonlinear analyses discussed herein will show that such a subtle data non-stationarity (i.e., small change in the degrees of freedom), which will also be shown to be representative of what heartbeat data are like, are difficult to interpret, especially for those linear or nonlinear algorithms which require data stationarity.

In an exemplary aspect, the methods, systems, and computer readable media provided can utilize electrophysiological data recorded by low-noise amplifiers and digitized at about 1000-Hz or higher. Further, the methods, systems, and computer readable media can use data simplification devices, such as R-R interval detectors and analytic algorithms. The analytic algorithm can be a PD2i data processing routine.

iii. Data Length Considerations

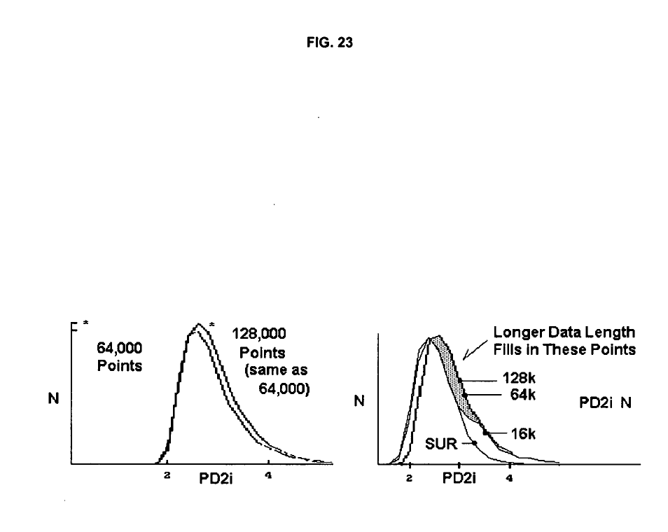

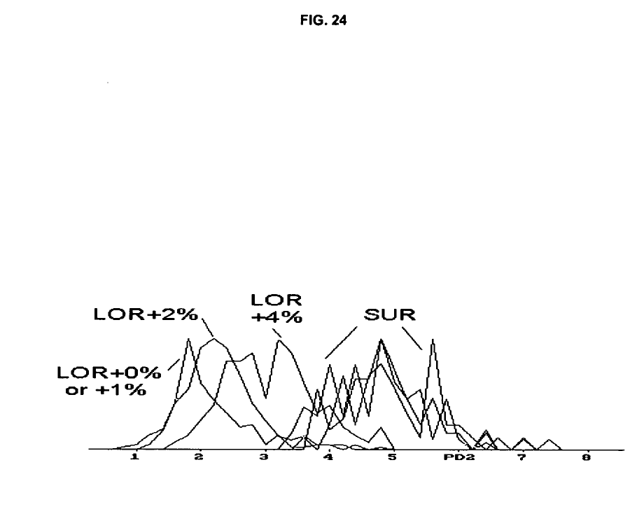

Data length (Ni) can be important in determining a nonlinear analytic result, therefore rules (Ni rules) have been developed that govern data length. If one samples data from a sleeping cat, FIG. 21, the distribution of the PD2i's does not change much beyond 64,000 data points (4.27 minutes). FIG. 23 (left) shows that the PD2i-distributions are essentially the same for 64,000 data points as they are for 128,000 data points; note that the peak of the histogram for 128,000 points is slightly higher than that for 64,000 to reveal both curves. The surrogates also overlap completely.

FIG. 23 (right) shows that increasing data length results in the PD2i's of the larger distributions becoming more unit-normal in appearance. The small skew to the right in all of the cases is due to noise content in the data caused by descretization error. Statistical correction for such skewness does not lead to any change in the interpretation of results, so this corrective step for statistical purposes is not warranted.

The PD2i's of the randomized-phase surrogate (SUR) are very normal in their appearance, as small noise content does not affect them as much. Since the t-test for significance requires unit-normal distributions, the higher data-point lengths are seemingly more valid for a t-test in surrogate testing than the 16,000 data point sub-epoch, but the latter is not statistically different from a normal distribution, so near-normal appearance, as in FIG. 23 (right), would seem to be satisfactory.

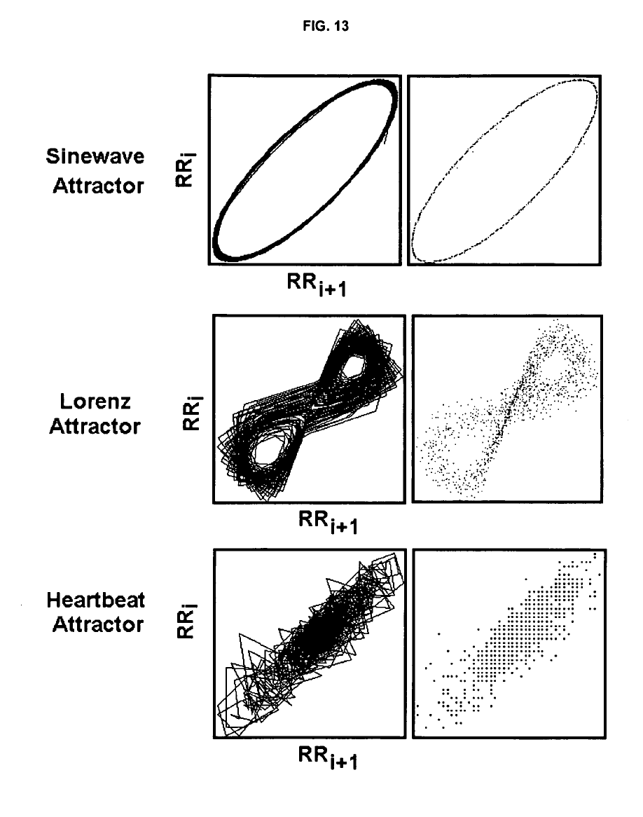

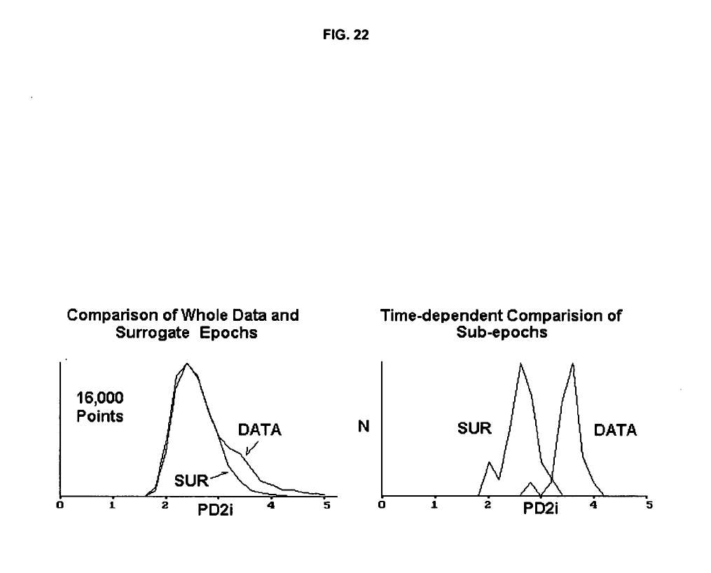

Swinney and associates (Wolf et al, 1985; Kostelich and Swinney, 1989) discussed data length requirements for determining the degrees of freedom of nonlinear attractors in phase space and came up with the rule, Ni>10 exp D2. This rule is commonly employed, but only works for attractors in which each lobe is often visited in phase space, as, for example, happens in the sine, Lorenz and heartbeat attractors seen in FIG. 13. The EEG attractor would not seem to obey this rule, for the lower mean (around 2.5) of the “total” sleep attractor (FIG. 22, left) would need around 64,000 data points to have a unit normal distribution for the PD2i values, whereas that for REM sleep attractor (FIG. 22, right), which is higher dimensional (around 3.5), has a unit normal distribution with only 1,250 data points. The latter, however, does obey the exponential rule for data length. The reason for this apparent discrepancy is that the Ni Rule requires data stationarity and only the brief REM sleep attractor is stationary and thus statistically different from its surrogate (randomized phase). The total sleep attractor is comprised of many different non-stationary subepochs and thus it is not different from its surrogate.

If the data being sampled are stationary and noise-free, then the exponential data-length rule, Ni Rule, (e.g., Ni>10 exp PD2i) can accurately determine the minimum data length in both generated and physiological data.

B. PD2i

The PD2i measures the time-dependent number of degrees of freedom of the regulators of the heartbeats that lie in the cerebral, autonomic, and intrinsic cardiac nervous systems. The PD2i can extend to other physiological time-series data within the capabilities of an ordinary technician to record. The algorithm and its embodiment have been disclosed under U.S. Pat. Nos. 5,709,214 and 5,720,294, both hereby incorporated by reference. The maximum PD2i indicates the maximum number of independent regulators (i.e., the number of cerebral, autonomic, and cardiac systems that contribute to its variability) and the minimum PD2i indicates the extreme of the time-dependent cooperation that exists among them. A minimum PD2i<1.4 indicates risk of arrhythmic death (Skinner, Pratt and Vybiral, 1993). A reduced maximum PD2i of heartbeats is indicative of early Alzheimer's disease, as disclosed (U.S. Patent Application No. 60/445,495, pending) and confirmed herein by FIG. 28, which shows results for both Dementia and Syncope patients. FIG. 28 shows the use of PD2i of heartbeats in defining dementia (Alzheimer's Disease) and cases of syncope.

i. Calculation of PD2i

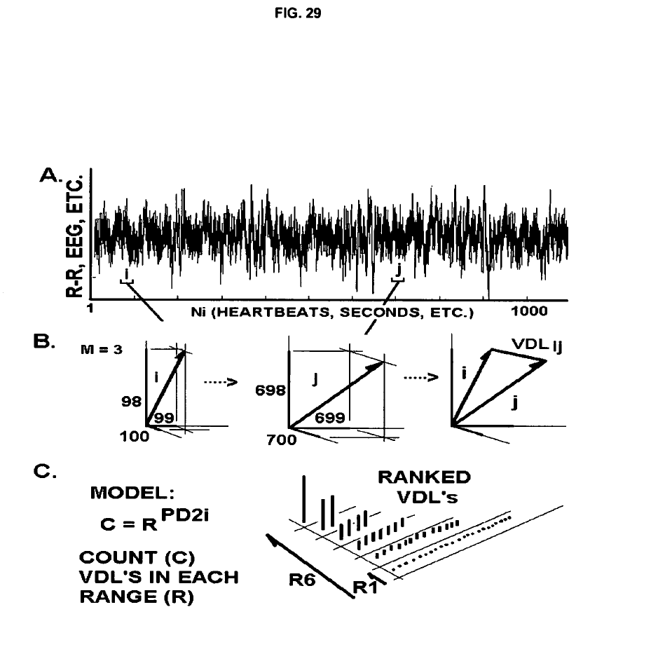

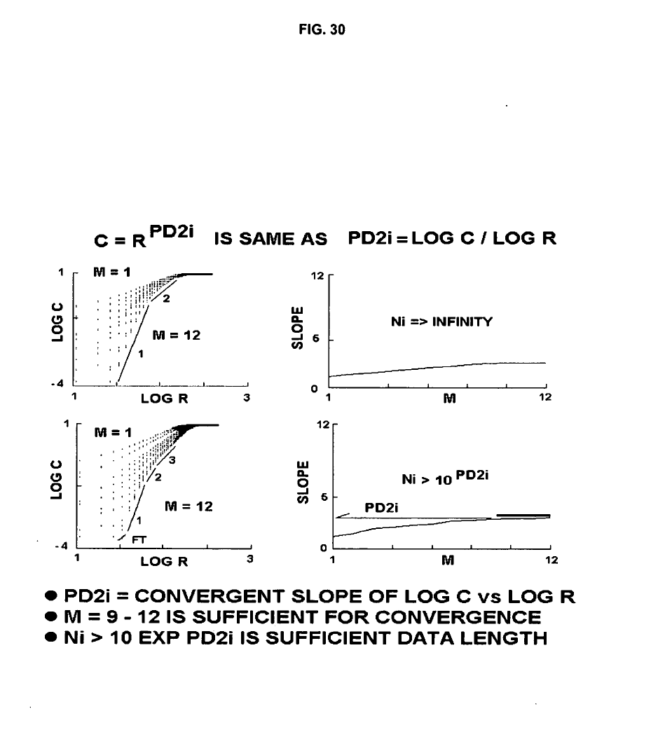

The calculation of the PD2i and the selection of its parameters, as previously disclosed in U.S. Pat. Nos. 5,709,214 and 5,720,294, is calculated as explained in FIGS. 29 through 31. FIG. 29 first shows how PD2i is calculated from vectors made from two samples of data points. Then FIG. 30 shows how the Correlation Integral, made from these vector difference lengths according to the mathematical model for PD2i (in the limit as Ni approaches infinity) appears for large data lengths and more realistic ones of finite data length. The Correlation Integral is the plot of the logC vs logR of the rank ordered vector difference lengths (VDL's) made at each of the embedding dimensions of M=1 to M=12. FIG. 30 also illustrates a Linearity Criterion (LC) for determining the linearity of the initial small log R slope (slope 1) that lies just above the floppy tail (FT) in more finite data lengths (lower left); the FT is caused by descretization error. Also illustrated is the Convergence Criterion (CC) which measure the lack of change of slope as embedding dimension is increases (lower right, horizontal bar).

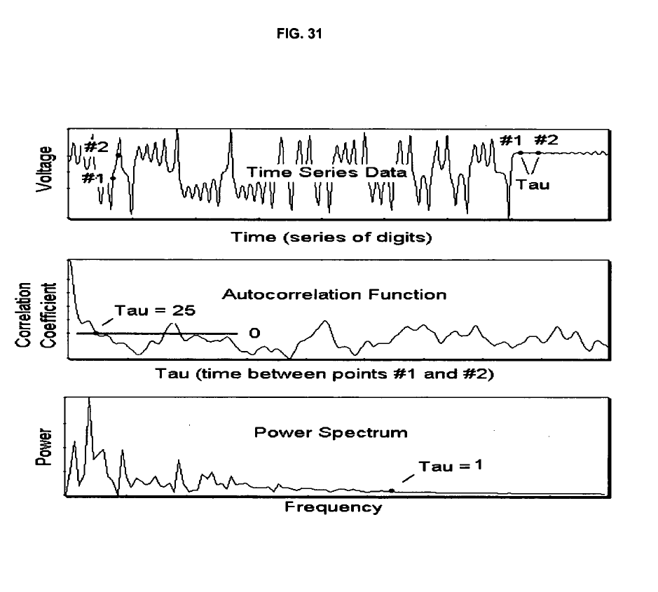

What is empirically observed for real and synthesized calibration data are the parameters that work well for PD2i analysis. The LC=0.30 exposes the unstable Floppy Tail that is due to finite data length combined with finite digitization rates. The segment of the first linear slope is restricted to 15% by a Plot Length (PL=0.15) parameter, with the minimum slope length being at least 10 data points in the log-log plot above the Floppy Tail (10-point Minimum criteria). The Convergence Criterion (CC=0.4) requires that the slope vs embedding dimension (M) converges, as it is the convergent slope value that defines each PD2i. Only PD2i values meeting these parameter requirements are the Accepted PD2i's. FIG. 31 shows how the parameter Tau is chosen and why Tau=1 was selected for heartbeat data.

FIG. 29 illustrates the calculation of the PD2i of a physiological time series (R-R, EEG, etc.) of data length, Ni. FIG. 29A. Brief paired samples of data (i, j), incremented for all i- and j-values, are used as coordinates for a multi-dimensional vector. FIG. 29B. The resultant vectors (i, j), shown for a three dimensional vector (M=3), are calculated and then the difference is calculated (VDLij). FIG. 29C. The mathematical model for the PD2i is: “C scales as R to the exponent PD2i as Ni (data length) approaches infinity, where C is the cumulative count of rank-ordered VDL's and R is a range over which the VDL's are counted; for example, for a smaller R (R=1) only the small VDL's are counted; for a larger range (R=6) all of the VDL's are counted; note that the number in each rank is usually larger for the small R values.

FIG. 30 illustrates calculation of PD2i as the convergent and restricted slope of the log C vs log R plot. Upper left: The plot of log C vs log R is made for each of the multi-dimensional vectors, from M=1 to M=12; M=12 means that 12 data points were used as coordinates to make a 12-dimensional i- or j-vector resultant. Upper right: The slope of the linear portion of the small log R plot for each dimension (M) is then made; note that as M increases beyond 9, the slope no longer increases (i.e., is convergent). Lower left: for finite data there is a floppy tail (FT) that is unstable and must be detected by the linearity criterion. The slope of the linear part just above the FT (slope segment 1) is then measured (parameter restricted to the first 15% of the whole plot). Its minimum length is 10 data points; otherwise it is rejected as a valid PD2i. Lower right: the plot of the restricted slopes are plotted vs M and found to be convergent for the higher M's (horizontal line), according to the Convergence Criterion. The criteria for the PD2i algorithm are: Tau=1, i.e., successive data points are used as coordinates; LC=0.3, i.e., the second derivative of the slope cannot vary more than plus or minus 15% of its mean, CC=0.4, i.e., the SD of M=9 through 12 cannot be more than plus or minus 20% of its mean; PL=0.15, i.e., the slope calculated is from the FT to 15% of the total number of data points in each plot, M=1 to M=12; Ni must be greater than 10 to the exponent PD2i (i.e., to calculate PD2i=0.0 to 3.0 accurately; as shown at the lower right, there must be at least 10 exp 3 data points in Ni (i.e., >1,000), that is, in the physiological data being analyzed.

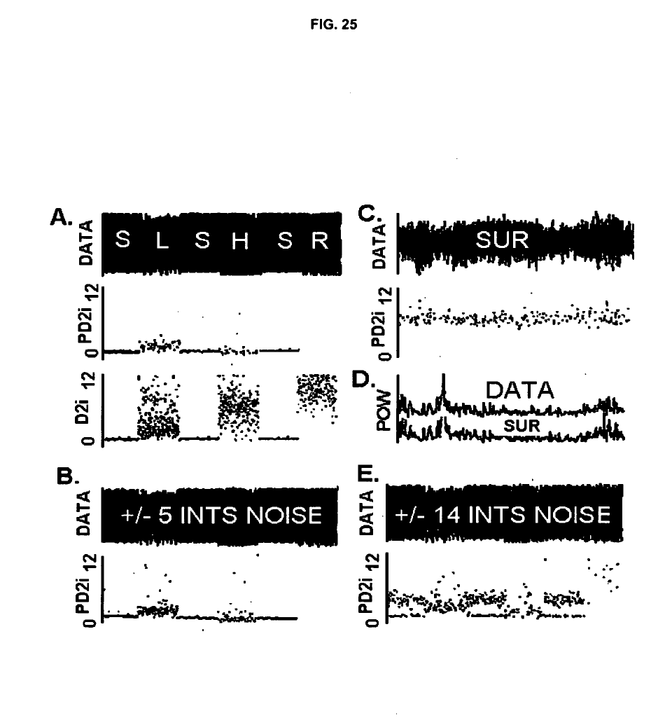

PD2i differs from D2 in that ePD2i is an estimate of D2, where E is an error due to the position i of the reference vector that is compared to all j-vectors to make the VDL's of the correlation integral. This error term (c) has a mean of zero, for all positions of the i-vector in the attractor. This means that as the i-position repeatedly loops through the attractor, mean PD2i will approach D2 in the limit, which empirically it does, with only 4% error, in the finite data of known mathematical origin shown in FIG. 25A.

FIG. 31 shows two ways to determine Tau, the number of data points skipped over to select those to be used in the ij-vector pairs as coordinates for making VDL's. Tau=1 means that successive points in the ij-samples of data are selected as coordinates for making the ij-vectors. Tau=2 means that every other data point is used, and so on. The same Tau must be used for all embedding dimensions, M=1 through M=12, to find the convergent slopes.

The upper panel in FIG. 31 shows two set of points, #1 and #2, drawn on Lorenz data. At the left, #1 and #2 are separated in time (data points) that have Tau=1 and, at the right, #1 and #2 are separated by Tau=10. If the Tau of #1 and #2 at the left was the same as that at the right, then the #2 point at the left would be past the upward spike in the data and be located at about the same value as #1 (i.e., on the y-axis). The points must therefore be close together, as at the left, to resolve the high frequency contributions toward dimensionality found in the whole data series.

The middle panel shows the Autocorrelation Function, where the Correlation Coefficient of the two points run through the entire data file is plotted versus its Tau. When Tau is zero, points #1 and #2 are always superimposed as they run through the data, so the Autocorrelation Function plot always starts off at a Correlation Coefficient=1.0. When the first zero crossing in the Autocorrelation Function is found, this means that points #1 and #2 are perfectly uncorrelated, that is, as the two points are incrementally run through the data to the find values to calculate the Correlation Coefficient. When the Correlation Coefficients are negative (below zero) they are negatively correlated, by various degrees, to a maximum of −1 (perfectly negatively correlated). For the Lorenz data shown in the upper panel, the first zero Correlation Coefficient in the Autocorrelation Function plot is at Tau=25. But this selection of Tau would not resolve the higher frequency contributions of the data shown in the upper panel.

Another way to select Tau is to first make the Power Spectrum of the data file, as shown in the lower panel of FIG. 31. When the higher frequency components stop contributing to the signal (and the PD2i), this implies a much smaller Tau, (see below), but one that will resolve the higher and lower frequencies. In the case of the Lorenz data this cutoff is at Tau=1. The peak Power implies Tau=25. That is, one quarter cycle of the frequency (Nyquist Frequency) of the Power peak is Tau=25, which implies that 100 data points are in the lower frequency sine wave of the Fourier transform at this frequency. There are 4 data points in the frequency of the Fourier transform at the indicated cutoff, where Tau=1. All frequency components, no matter what their relative powers are, contribute equally toward the measurement of the degrees of freedom (i.e., PD2i, expressed in dimensions).

For limited numbers of finite data length, it is always better to use a small Tau, as it will enable the nonlinear detections of the dimensions of the attractors for both the low and high frequency lobes. In the data shown in the upper panel a Tau=1 would detect dimensional contributions of both the high frequency spikes at the left and the low frequency (flat) segment at the right; Tau=10 or 25 would only detect the latter. Note that Tau=1 revealed the attractors for the sine, Lorenz and heartbeat data shown in FIG. 13. Tau=1 can thus be selected for heartbeat analysis, as this can optimally display the attractors whose dimensions are calculated by PD2i.

A feature that distinguishes the PD2i algorithm from the D2i algorithms is to restrict the length of the initial slope-1 linear scaling region that lies above the unstable Floppy Tail. This provides for the accuracy of the PD2i algorithm in non-stationary data (FIG. 25A). Only ij-vector differences made from the same species of data will create the very small vector difference lengths (VDL's). Those VDL's in which the i-vector and j-vector are each in different species of data (e.g, one is in sine data and the other in Lorenz data, as in the non-stationary data shown in FIGS. 11 and 25A) tend to be larger than those made when the i- and j-samples are both in the same species. This is both mathematically true and empirically supported by marking and observing the VDL's in the Correlation Integral.

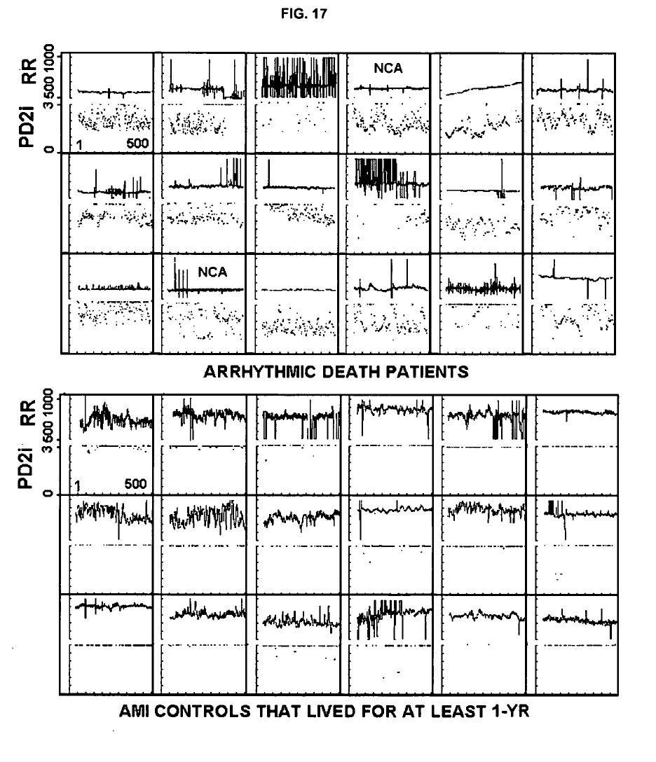

It has been empirically determined that the 15% restriction on the Plot Length, with a minimum of 10 points above the Floppy Tail, works well in both known non-stationary data (4% error, FIG. 25A) and in physiological data whose outcomes are known (FIG. 17). This restriction works well even if noise of small amplitude is in the data. For example, the noise will make small VDL's and thus contaminate the initial part of the logC vs logR linear scaling region above the Floppy Tail, a slope which is the PD2i. This noise-related contribution to the slope will be additive to that of the small logR values derived from the attractor and thus will slightly increase or boost the mean PD2i. But this small amount of noise can be dealt with algorithmically.

A computational technique incorporated in the PD2i algorithm is to set the very small slopes to zero, as these are likely to be caused entirely by noise and not by any signal with variations. Setting the slopes less than 0.5 to 0.0 provides for the ±5 integer (msec) noise tolerance level of the PD2i algorithm, in which ±5 integers of random noise can be added to the larger amplitude data without significantly increasing the PD2i values (FIG. 25B).

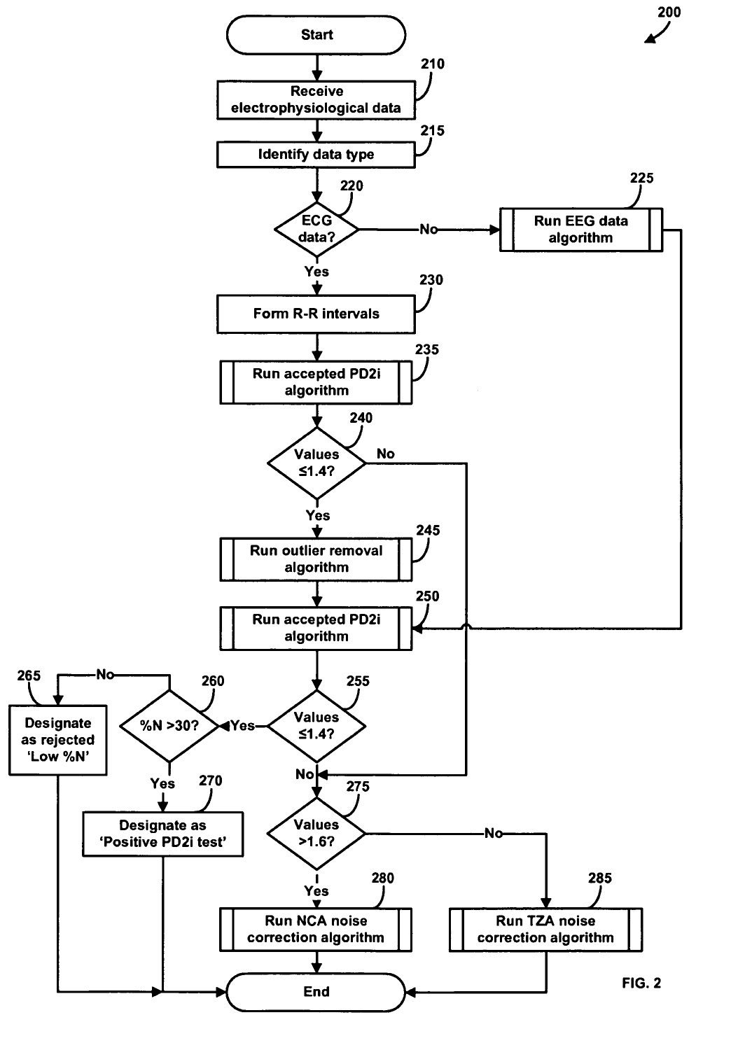

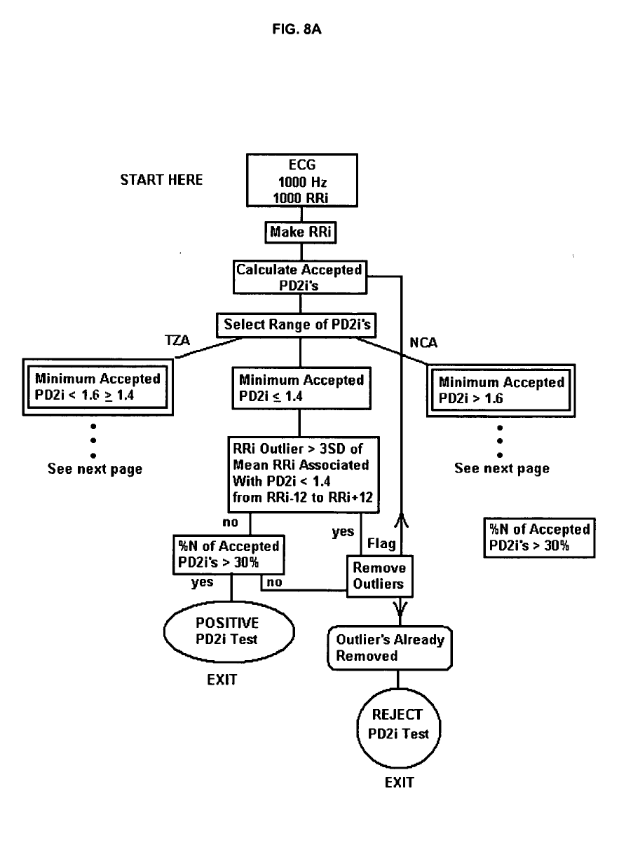

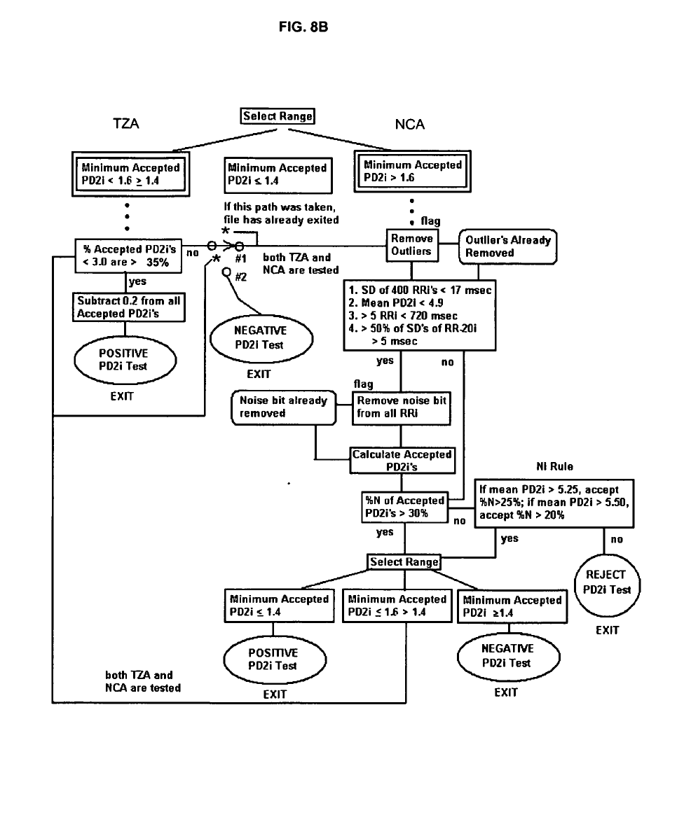

Another technique to address the boosted mean PD2i is to use the Noise Consideration Algorithm (NCA) and the Transition Zone Algorithm (TZA) described herein (FIGS. 2 and 8 A-C).

ii. PD2i and Noise

As described herein, noise can get into R-R interval data from physiological sources (e.g., atrial fibrillation or high arrhythmia rates), errors in the RR-detector (small R-waves confused with T-waves), broken equipment (broken leads that produce artifacts), or poor data-acquisition technique (e.g., failing to properly instruct the patient or behaviorally control the environment). Also noise can get into EEG data from physiological sources (e.g., non-REM sleep is not different from its surrogate), poor equipment (e.g., not recording with proper bandpass or digitization rate) or poor data-acquisition technique (ambient noise, lack of a controlled environment). All of these sources of noise must be dealt with to stay within the range of the noise tolerance level of the PD2i algorithm, as judged by % N of accepted PD2i's, otherwise the data must be excluded from study on an a priori basis because of its noise content. The NCA, TZA and removal of outliers are all noise-reducing algorithms for dealing with small amounts of unavoidable noise that would otherwise lead to their exclusion from study. The NCA has been disclosed in U.S. patent application Ser. No. 10/353,849 hereby incorporated by reference. The TZA is disclosed herein.

a. % N

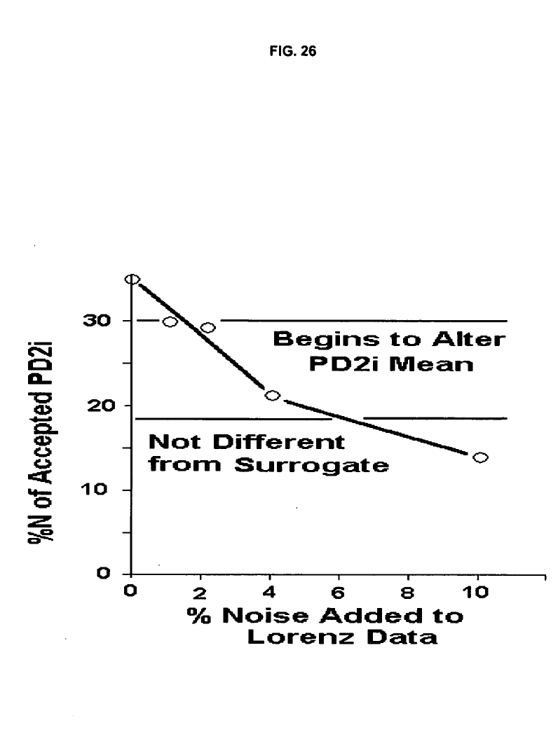

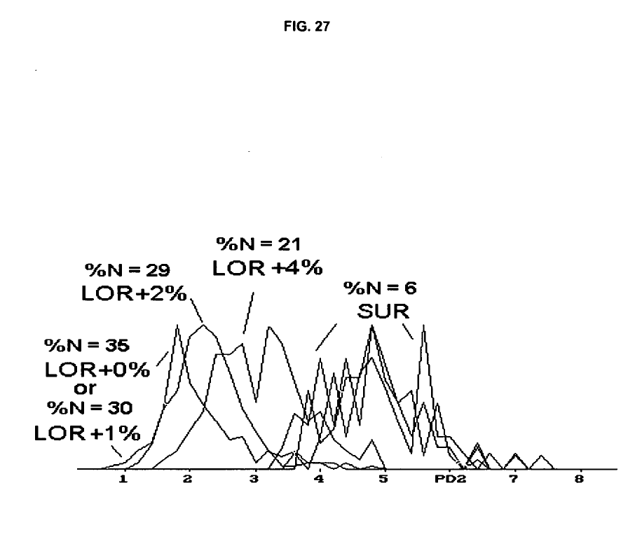

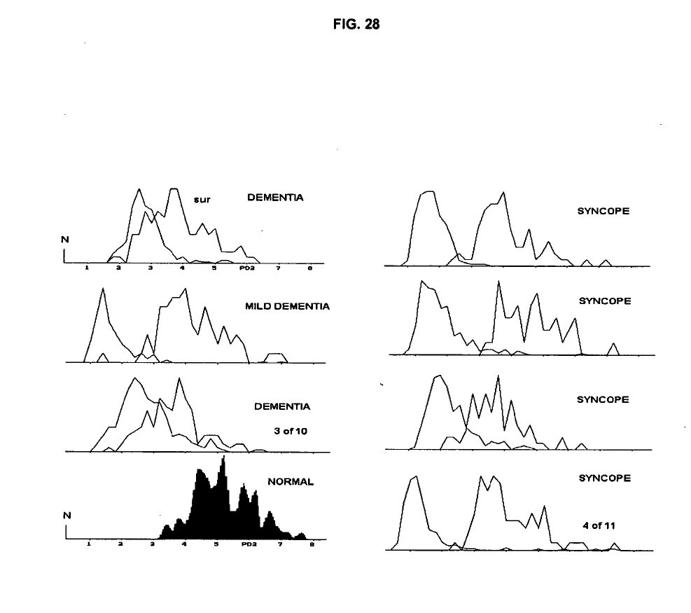

A method to apply to electrophysiological data, to assure that noise in the electrophysiological data is not leading to spurious calculation by nonlinear algorithms, is to test the null hypothesis that the data are the same as filtered random noise (i.e., by the Randomized Phase Surrogate Test). If the result of the experimental data are statistically different from that of their surrogate, using the same analytic algorithm on both data types, then the null hypothesis is rejected—i.e., the data are not filtered noise. FIGS. 26 and 27 show that systematically adding noise to noise-free Lorenz data reduces the % N (ratio of accepted PD2i's to all PD2i's) and marches the mean PD2i of the data toward that of the surrogate. At % N>30, the noise does not alter the distribution of the PD2i scores, but at % N<30, it does. This constitutes mathematical evidence that % N>30 should be a criterion for adequately sampled data. If the data fail to meet the Ni-rule (Ni>10 exp PD2i) this will appear as noise and thus cause rejection by % N. It has been empirically observed in the 340 ER patient database that if mean PD2i is greater than 5.25 (requiring 500,000 RR-intervals, which would take 125 hours to record), then % N of 25% is acceptable, and if mean PD2i is greater than 5.75, then % N of 20% is acceptable. That is, there were no low dimensional PD2i in these files, but the % N was not acceptable because of the high mean PD2i and inappropriate Ni, so adjustments to the % N should be allowed, as they were all found to be True Negative data files. By way of example, parameters for % N can be % N<30, except when there are no PD2i's less than 1.6, when mean PD2i is greater than 5.0, 5.25 or 5.75, indicating % N>29%, % N>25% and % N>20%, respectively, as acceptable. Small amounts of noise may still remain the data that require additional algorithmic handling for nonlinear analyses.

The ratio of the number of Accepted PD2i's to the total possible PD2i's (% N) is nonlinearly correlated with the amount of noise in the data. A reason PD2i's are rejected is because of failure to meet the criteria for the correlation integral. FIG. 26 shows the nonlinear relationship of % N Accepted PD2i's to % Noise Content for Lorenz data (1200 data points). Noise (random) is systematically added to the noise-free data. For values of % N at or above 30%, the noise content does not alter the mean PD2i (upper horizontal line). For values below 19% N the noise content of the data is too large to enable the rejection of the null hypothesis that the PD2i distribution of the data is the same as that for filtered noise (i.e., its randomized-phase surrogate).

FIG. 27 shows the same effect as in FIG. 26, but with the noise content (LOR+% noise) and % N shown for the PD2i distributions. Because adding 1% noise does not alter the PD2i distribution at all (completely overlapped LOR+0% and LOR+1%), a % N of 30 seems to be acceptable. But adding 2% noise causes a 0.5 degrees of freedom shift of the entire PD2i distribution to the right, including the lowest values in the left-hand wing. Adding still more noise (4%), although it is still marginally statistically significantly different from its surrogate, results in a distribution that is broader, with a peak different from the mean, and is farther shifted toward its surrogate.

% N>30% can thus be a measure of the stability of the PD2i distribution, including the lowest values, and whether or not the distribution will be statistically significantly different from that of its randomized-phase surrogate.

b. Removing Outliers (Non-Stationary Artifacts)

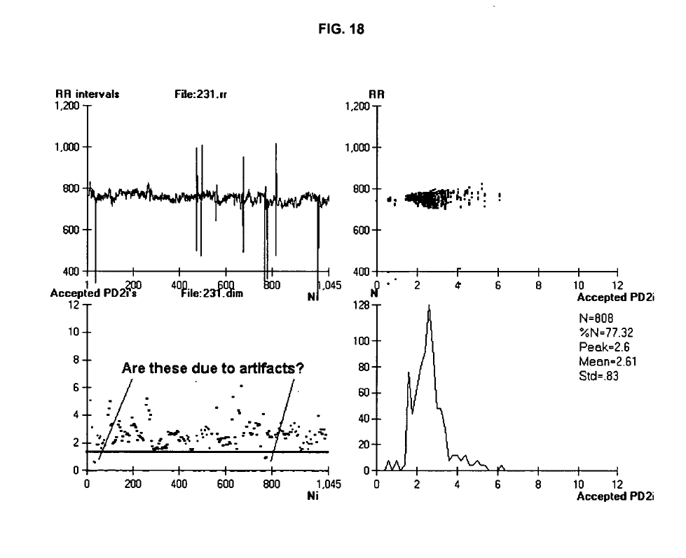

It is common practice to remove outliers in a data series where values greater than a deviation threshold (for example, 3 Standard Deviations) exist, as these are thought to be non-stationary events (i.e., noise). Interpolations over them (linear spline or “splining”), instead of removing them, maintains correlations in time. In nonlinear analyses using the correlation integral (D2, D2i, PD2i), these singular points in the data are usually rejected by linearity and convergence criteria (discussed herein), but if more than a few are present, scaling can occur in the correlation integral that produces spurious values, as seen in FIG. 18. FIG. 18 shows nonlinear results (PD2i) when the physiological data (RR intervals) contain artifacts (arrhythmias, movement artifacts). The artifacts are the large spikes seen in the RR Interval trace (upper left). The corresponding PD2i scores are shown in the lower left quadrant. The plot of RR Interval vs PD2i is shown at the upper right and the PD2i histogram is shown in the lower right quadrant. Some of the PD2i's which have movement artifacts or arrhythmias contaminating the reference vector (large spikes) are rejected, but not all.

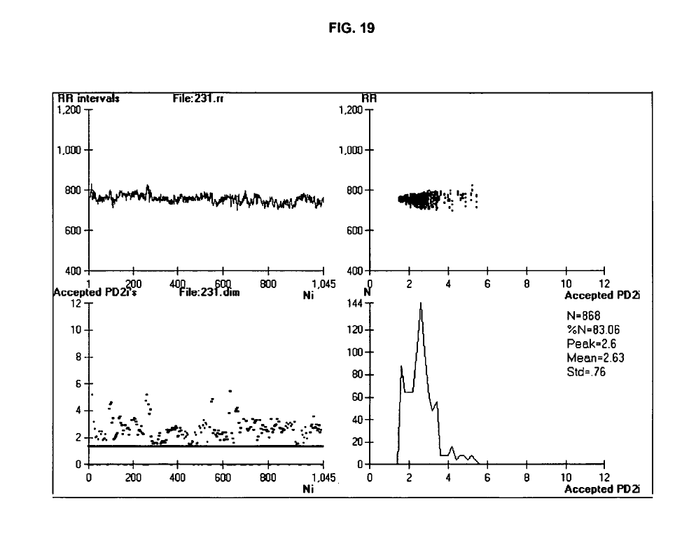

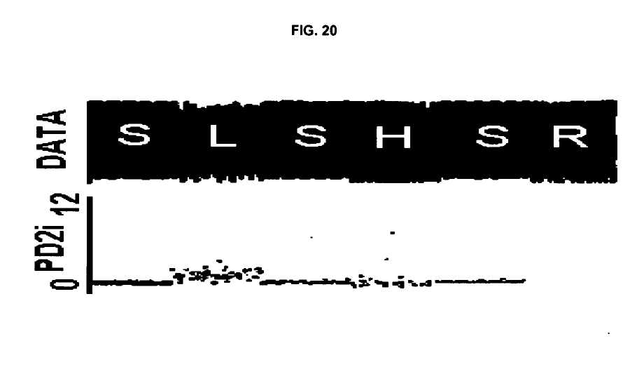

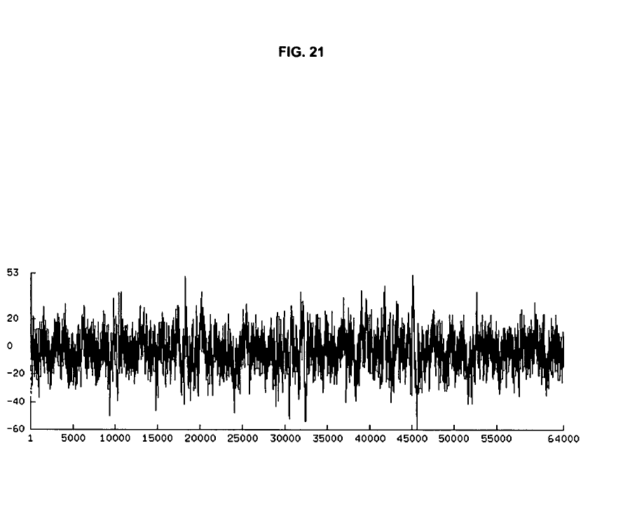

If the artifacts are removed by an interpolation spline (linear interpolation), then the low PD2i values are eliminated, as shown in FIG. 19. The outliers can be modified by overwriting them with a linear spline that reaches backward in time by one point and forward in time by one point (i.e., uses the i−2 values and i+2 values to construct the linear interpolation values to overwrite i−1 to i+1). FIG. 19 illustrates the same data file and results as in FIG. 18, but the artifacts have been removed by a linear spline that overwrites them. The relative importance of such artifacts should be considered and routinely removed from heartbeat data, especially if the data spuriously produce PD2i scores are below the TZA threshold, discussed herein.

c. NCA and NCA Criteria

According to exemplary aspects, the NCA (noise consideration algorithm) examines low level noise at high magnification (e.g., y axis is 40 integers full scale, x-axis is 20 heartbeats full scale) and determines whether or not the noise is outside a predetermined range, for example, whether the dynamic range of the noise is greater than ∀5 integers. If it is, then a noise is removed from the data series by dividing the data series by a number that brings the noise back within the range of ∀5 integers. For example, the data series may be divided by 2, removing a noise bit.

Since the linear scaling region of the correlation integral, calculated at embedding dimensions less than m=12, will have slopes less than 0.5 when made from low-level noise (e.g., with a dynamic range of ∀5 integers), it is impossible to distinguish between low-level noise and true small slope data. Conveniently, since slopes less than 0.5 are rarely encountered in biological data, the algorithmic setting of any slopes of 0.5 or less (observed in the correlation integral) to zero will eliminate the detection of these small natural slopes, and it will also eliminate the contribution of low-level noise to the PD2i values. It is this “algorithmic phenomenon” that explains the empirical data and accounts for the lack of effect of noise within the interval between −5 and 5 when added to noise-free data. Noise of slightly larger amplitude, however, will show the noise-effects expected to occur with nonlinear algorithms.

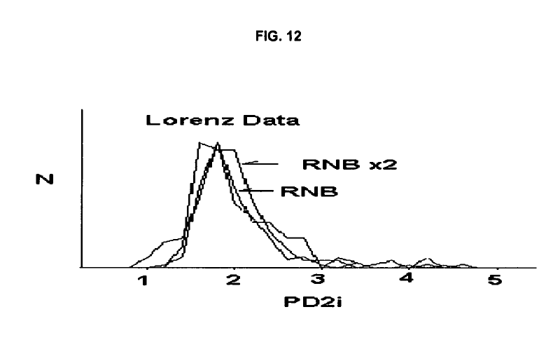

Removing a noise-bit cuts the noise in half, as is shown in FIGS. 12 (Lorenz data) and 14 (RR data), and thus brings the slope values back into their non-boosted state (i.e., the noise is now less than the noise tolerance level). But doing this for every data file is unwise, as it may cause the PD2i algorithm to overlook the small logR values from the physiological data that may be important in some cases. In other words, there must be some reason for suspecting that the file contains noise before a noise-bit is removed from the data.

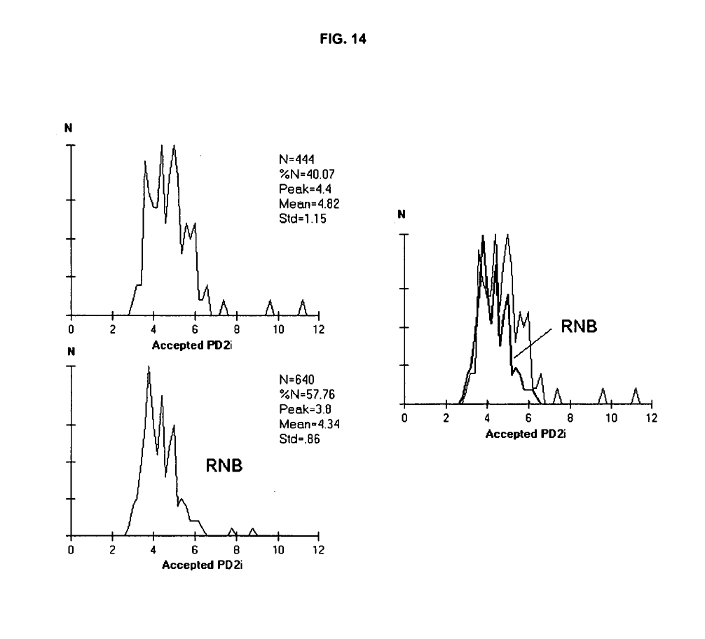

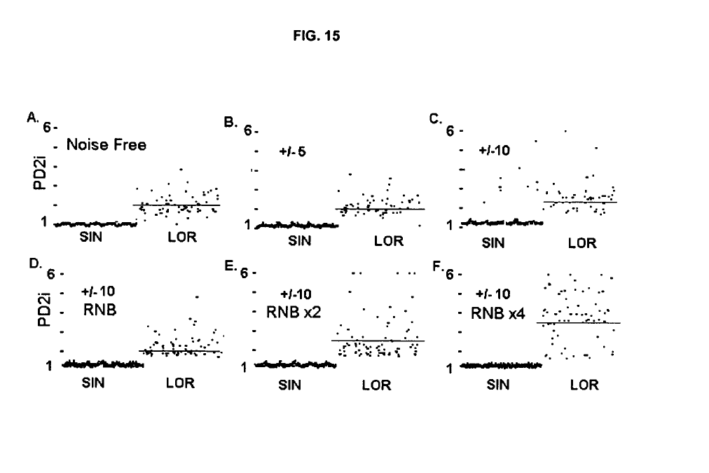

Noise is usually quantified as a percentage of the signal content. Filtering out noise also filters out part of the signal, which in nonlinear analyses could potentially lead to spurious results. By removing a bit (e.g., dividing the amplitude of the signal by 2), the noise in the signal is also reduced by half. FIG. 12 shows that removing a bit does not significantly alter the mean or distribution of a nonlinear measure, the PD2i. The effect of removing a “noise” bit (RNB) on the distribution of the nonlinear measure of Lorenz data by the Point Correlation Dimension (PD2i) is shown. Reducing the amplitude of the Lorenz data by half (RNB) does not significantly alter its distribution compared to the original unaltered signal. In contrast, removal of two bits (dividing the amplitude by 4) does alter the distribution by widening it in the middle. Removing 2 bits, changes the distribution by flattening the middle part and widening the wings of the histogram. This is undesirable, as it removes too much signal. Removing a single bit from RR data (FIG. 14) has no effect on the smaller PD2i values, including the minimum PD2i.

The NCA can be run in “almost-Positive” PD2i cases (i.e., Negatives ones with minimum PD2i having a low dimensional excursion close to the separatrix), as defined in the paragraph below. Removing a noise-bit will have no affect in obviously Negative files with large R-R Interval variability. Removing a noise-bit in already Positive PD2i cases is not required, as it would only make them more Positive.

Examples of NCA criteria that can be used in determining boosted noise content in the almost-Positive RR-interval data include, but are not limited to: 1) the R-R Interval data are somewhat “flat,” with little heart rate variability (i.e., the SD of 400 successive R-R Intervals, of at least one segment, is less than 17 msec); 2) the mean PD2i is below the usual normal mean of 5.0 to 6.0 (i.e., the mean PD2i<4.9); 3) the R-R Intervals go to low values, indicating high heart rate, at least once in a 15-minute data sample (i.e., 5 R-R Intervals<720 msec), and 4) there actually is a small amount of noise in the data (i.e., more than 50% of the running windows of 20 RR-Intervals have an SD>±5).

d. TZA and TZA Criteria

If nonlinear measures of physiological data are on a continuous scale and are used to stratify the analytic outcomes above and below a separatrix (e.g., to predict risk of arrhythmic death), then a transition zone algorithm (TZA) can be required to better separate the outcomes into the two strata. For transient physiological changes in the results (e.g., PD2i scores), which represent non-stationary events, one can adjust a TZA threshold by the actual outcomes (e.g., arrhythmic death events or no arrhythmic deaths) in a test data set. This test-retest adjusting can first determine the position of the TZA threshold in one data-set, and then use the TZA threshold in a subsequent data-set. A problem with this method is that a transient low-dimension excursion of the PD2i may occur in either the test or re-test, which may approach an infinitely thin separatrix or criterion level, but fail to reach it because the nonlinear scores are slightly elevated by a small amount of noise in the data. Thus a noise correction factor is needed.

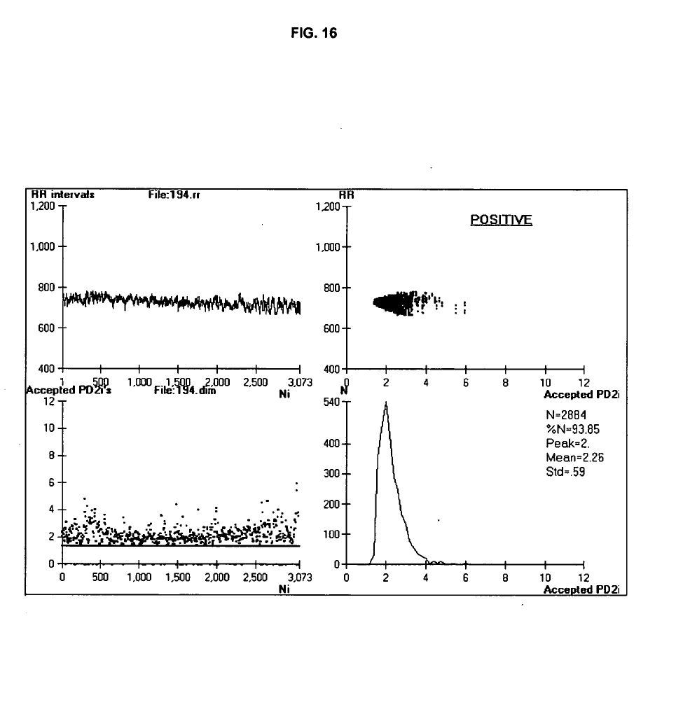

FIG. 16 shows an example of a subject with multiple low-dimension excursions of PD2i into a transition zone that lies just above the separatrix (horizontal line, lower left). The separatrix can be for example, 1.4. The transition zone can be between 1.4 and 1.6. There are multiple PD2i scores in the transition zone between 1.4 and 1.6, when the a priori separatrix has been set a 1.40. The subject's scores in FIG. 16 might be slightly elevated by noise content. Once a score is determined to be within the transition zone, the score can be lowered by a small number of dimensions to compensate for the small elevation caused by the small amount of noise. The number of dimensions can be, for example, 0.2.

In a study of 320 cardiac patients presenting in the ER, there were 20 subjects that had PD2i scores in the transition zone between 1.4 and 1.6 dimensions, where 1.4 was the a priori separatrix determined in a previous study. Of these, 3 had Arrhythmic Death (AD) outcomes and were True Positives (TP); 16 had non-AD and were True Negatives (TN); and 1 had non-AD, and was a False Positive (FP). The problem is how to separate the three AD's from the 17 non-AD's when PD2i scores lie in the small Transition Zone just above the a priori separatrix.

If one examines all of the PD2i scores in all of the 320 patients, then it becomes quite apparent that the AD's have many PD2i's less than 3.0 that and the non-AD's do not. This effect is illustrated in FIG. 17, where the AD's are compared to their non-AD controls, each of whom had an acute myocardial infarction but did not manifest AD in a 1-year follow-up period. In the upper portion of FIG. 17, RR and PD2i data are shown from 18 patients who died of defined sudden arrhythmic events (AD) within the 1-year of follow-up; the majority died within 30 days. In the lower portion of FIG. 17, similar data are shown from 18 controls, each of whom had a documented acute myocardial infarction (AMI) and lived for at least the 1-year of follow-up. These outcome results suggest that one could simply count the PD2i values below 3.0 and find statistically significant results. In fact, when this is done, a posteriori, the Sensitivity and Specificity are each at 100% (p<0.001). But note in the individual patient cells in the top half of this figure that there are many transient low-dimensional excursions. Also note that for the Non-AD patients there are relatively few single points that dip into the 0 to 3.0 zone.

Another consideration is that if one were to use the number of PD2i's over the 10- to 15-minute period of the ECG recording (a stochastic measure), one must then presume data stationarity during this interval, which is not the case, as the dipping of the low-dimensional PD2i excursions are indicative of non-stationary events (i.e., the degrees of freedom are changing). So, the minimum of the low dimensional excursion is a criterion for the PD2i nonlinear measure, for both practical and mathematical reasons.

To resolve the dilemma of the transient low-dimensional PD2i scores in the transition zone, which all could be slightly elevated because of small noise content, it is permissible to use an independent stochastic measure of the PD2i population as a criterion for assessing noise content in all of them and then adjusting the transient PD2i scores accordingly.

When a Transition Zone Algorithm (TZA) is used as a noise correction factor, that incorporates a 35% threshold of accepted PD2i less than 3.0, and which is independent of the Noise Consideration Algorithm discussed herein, in which a noise bit may or may not be removed, then all of the minimum PD2i scores in the transition zone break into the correct PD2i prediction of AD. This is a highly statistically significant breakout using non-parametric statistics (binomial probability, p<0.001). Such an a posteriori noise-correction factor may thus be commonly used when data contain a small amount of noise.

In sum, TZA criteria include, but are not limited to, 1) there must be at least one PD2i value in the Transition Zone (PD2i>1.4, but PD2i≦1.6); 2) The mean PD2i must be markedly reduced (less than 35% of Accepted PD2i<3.0). If these criteria are met, then the PD2i values can be reduced by 0.2 dimensions.

### III. Exemplary Aspects

A. General Aspects

In one aspect, illustrated in FIG. 37, provided are automated methods of compensating for small amounts of unavoidable noise associated with electrophysiological data for more effectively predicting a biological outcome, such as arrhythmic death, steps of the methods comprising, at step 3701, defining a plurality of intervals, such as R-R intervals, having associated interval data, wherein each interval is associated with a time duration between consecutive portions of a trace, such as an ECG or an EEG trace, corresponding to a first portion of the electrophysiological data, analyzing the plurality of intervals using a data processing routine, such as the PD2i, to produce dimensional data at step 3702, and removing at least one extreme value, such as an outlier, from the interval data when the dimensional data is less than a first threshold at step 3703. The first threshold can be about 1.4. Removing at least one extreme value can produce refined dimensional data. The methods can further comprise analyzing the refined dimensional data using a data processing routine, such as the PD2i, to produce acceptable dimensional data at step 3704, and predicting an arrhythmic death when the acceptable dimensional data is below a second threshold and above a qualifying condition at step 3705. The second threshold can be about 1.4. The qualifying condition can be when a % N of accepted or refined dimensional data is above a third threshold. The third threshold can be about 30 percent. The qualifying condition can be expressed as % N>30%, wherein % N is the percentage of PD2i's that were accepted.

The step of removing the at least one extreme value can comprise identifying an outlying interval within the plurality of intervals, wherein the outlying interval is outside a deviation threshold, defining a linear spline for the outlying interval, and overwriting the outlying interval with the linear spline. The deviation threshold can be, for example, 3 standard deviations.

The methods can further comprise a noise correction algorithm. The noise correction algorithm can be, for example, an NCA, a TZA, and the like.

The methods can further comprise determining whether the electrophysiological data are either electroencephalogram data or electrocardiogram data. If the electrophysiological data are EEG data, the methods can further comprise an EEG data algorithm. The EEG data algorithm can comprise selecting a linearity criterion, selecting a plot length, selecting a tau, selecting a convergence criterion, and defining the accepted PD2i values in response to selecting the linearity criterion, the plot length, the tau, and the convergence criterion.

In another aspect, illustrated in FIG. 38, provided are automated methods of reducing or compensating for small amounts of noise associated with electrophysiological data for more effectively predicting a biological outcome, such as arrhythmic death, comprising, at step 3801, forming R-R intervals from the electrophysiological data, defining accepted PD2i values from the R-R intervals at step 3802, and determining whether the accepted PD2i values are less than a first threshold value at step 3803. The first threshold can be about 1.4. The methods can further comprise removing R-R interval outliers when the accepted PD2i values are less than the first threshold value at step 3804, defining refined accepted PD2i values in response to removing the R-R interval outliers at step 3805, determining whether either the accepted PD2i values or the refined accepted PD2i values are below a second threshold at step 3806, and predicting an arrhythmic death when either the accepted PD2i values or the refined accepted PD2i values are below the second threshold and above a first qualifying condition at step 3807. The second threshold can be about 1.4. The first qualifying condition can be a % N of accepted or refined dimensional data above a fifth threshold. The fifth threshold can be about 30 percent.

The methods can further comprise classifying the electrophysiological data as electroencephalogram data.

The methods can further comprise determining whether either the accepted PD2i values or the refined accepted PD2i values are in a transition zone. The methods can accomplish this by determining if the accepted PD2i values or the refined accepted PD2i values are above a third threshold when it is determined that either the accepted PD2i values or the refined accepted PD2i are not below the second threshold. The third threshold can be about 1.6. The methods can further comprise applying a transition zone correction (TZA) when it is determined that either the accepted PD2i values or the refined accepted PD2i values are not above the third threshold, thereby determining that the accepted PD2i values or the refined accepted PD2i values are in the transition zone.

Applying the transition zone correction can further comprise determining whether either the accepted PD2i values or the refined accepted PD2i values meet the TZA criteria. The methods can accomplish this by determining if the accepted PD2i values or the refined accepted PD2i values are above the first qualifying condition. The first qualifying condition can be a % N of accepted or refined dimensional data above a fifth threshold. The fifth threshold can be about 30 percent. The methods further comprise determining whether a second qualifying condition for either the accepted PD2i values or the refined accepted PD2i values is less than a fourth threshold. The second qualifying condition can be a percentage of accepted or refined PD2i values less than about 3. The fourth threshold can be about 35 percent. The methods still further comprise subtracting an offset from either the accepted PD2i values or the refined accepted PD2i values, and predicting the arrhythmic death in response to subtracting the offset. The offset can be, for example, 0.2.

The methods can further comprise applying a noise content (NCA) correction when it is determined that either the accepted PD2i values or the refined accepted PD2i values are above the third threshold.

In yet another aspect, illustrated in FIG. 39, provided are automated methods of reducing noise associated with electrophysiological data for more effectively predicting a biological outcome, such as arrhythmic death, steps of the methods comprising, at step 3901, associating the electrophysiological data with a first data type, such as an ECG/EKG or EEG data type, forming R-R intervals from the electrophysiological data at step 3902, defining accepted PD2i values from the R-R intervals at step 3903, determining whether the accepted PD2i values are less than a first threshold value at step 3904, and removing outliers when the accepted PD2i values are less than the first threshold value at step 3905. The first threshold can be about 1.4. The methods can further comprise defining refined accepted PD2i values in response to removing outliers at step 3906, determining whether either the accepted PD2i values or the refined accepted PD2i values are below a second threshold at step 3907 and predicting an arrhythmic death when either the accepted PD2i values or the refined accepted PD2i values are below the second threshold and above a qualifying condition at step 3908. The second threshold can be about 1.4 and the qualifying condition can be when a percentage N of accepted or refined dimensional data is above a fourth threshold. The fourth threshold can be about 30 percent.

The methods can still further comprise determining whether either the accepted PD2i values or the refined accepted PD2i values are above a third threshold when it is determined that either the accepted PD2i values or the refined accepted PD2i are not below the second threshold at step 3909, applying a transition zone correction when it is determined that either the accepted PD2i values or the refined accepted PD2i values are above the third threshold at step 3910, and applying a noise content correction when it is determined that either the accepted PD2i value or the refined accepted PD2i value is below the third threshold at step 3911. The third threshold can be about 1.6.

Applying a transition zone correction can comprise subtracting an offset from either the accepted PD2i values or the refined accepted PD2i values and predicting the arrhythmic death in response to subtracting the offset. The offset can be, for example, 0.2.

Applying a noise content correction can comprise removing an outlier greater than a predetermined number of standard deviations of the R-R intervals. The predetermined number of standard deviations can be 3. The noise content correction can further comprise determining if the R-R intervals meet a predetermined number of NCA criteria, removing a noise bit from each R-R interval, if the predetermined number of NCA criteria are met, re-defining accepted PD2i values from the R-R intervals, and predicting the arrhythmic death in response to the redefined PD2i values. Removing a noise bit can comprise dividing R-R interval amplitude by 2. NCA criteria that can be used in determining noise content include, but are not limited to: 1) the R-R Interval data are somewhat “flat,” with little heart rate variability (i.e., the SD of 400 successive R-R Intervals, of at least one segment, is less than 17 msec); 2) the mean PD2i is below the usual normal mean of 5.0 to 6.0 (i.e., the mean PD2i<4.9); 3) the R-R Intervals go to low values, indicating high heart rate, at least once in a 15-minute data sample (i.e., 5 R-R Interval<720 msec), and 4) there actually is a small amount of noise in the data (i.e., more than 50% of the running windows of 20-R-R Intervals have an SD>±5).

B. Detailed Aspects

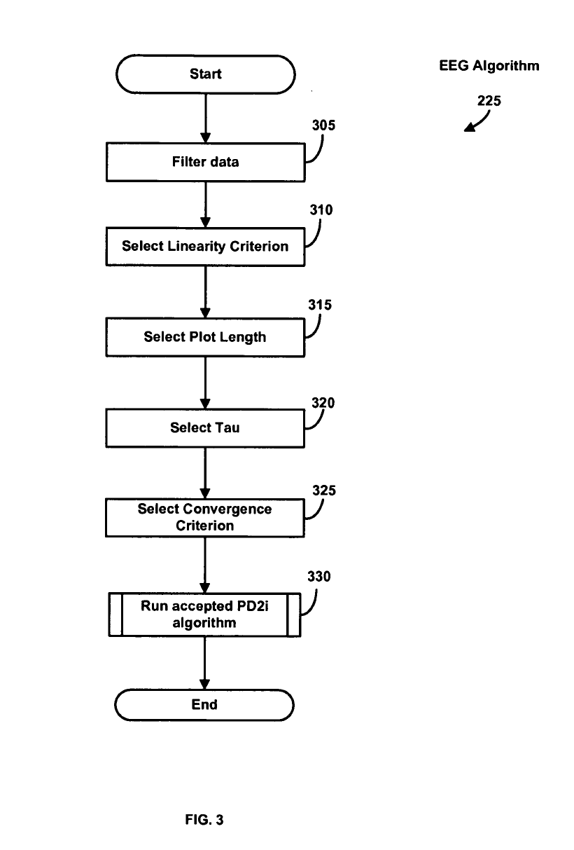

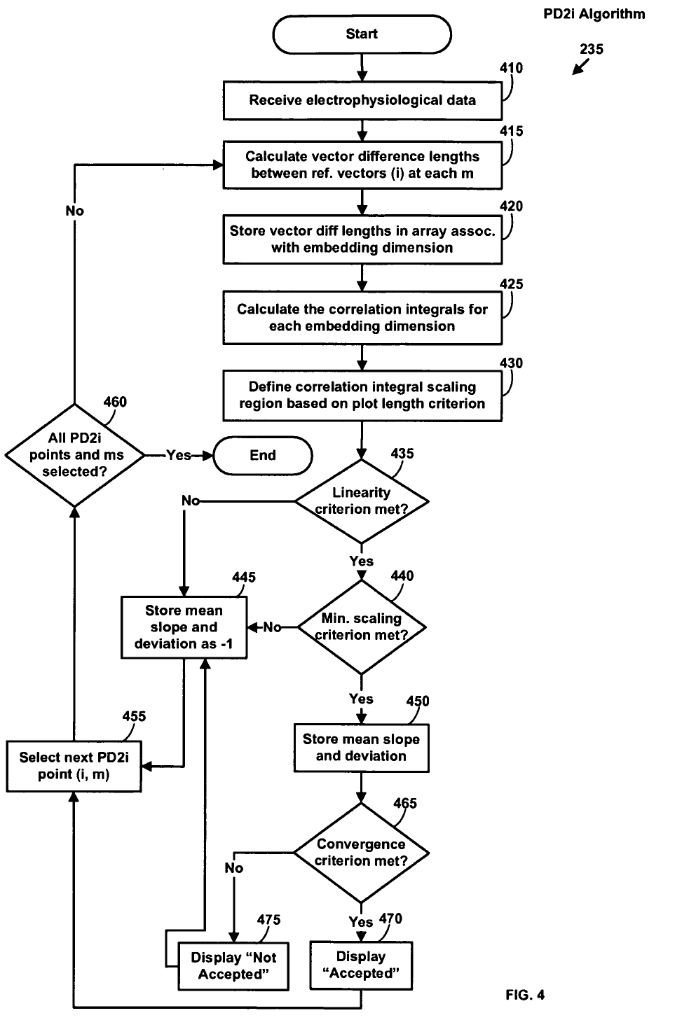

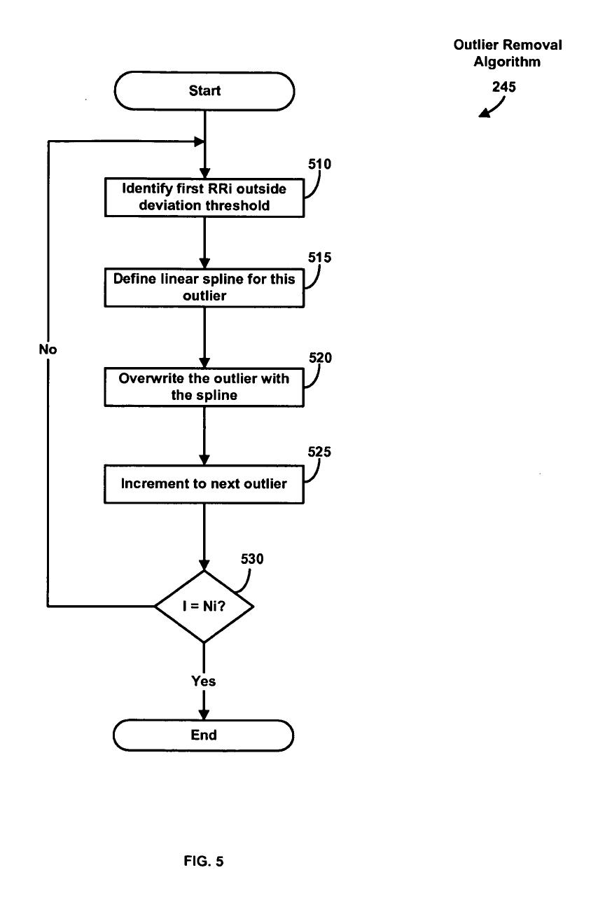

FIG. 2 illustrates another aspect of the present methods. The method begins at step 210. In step 210, the method receives electrophysiological data, for example EEG or ECG data. Step 210 is followed by step 215. In step 215, the type of electrophysiological data is identified. Step 210 is followed by the decision step 220. In step 220, the method determines if the data is ECG data. If it is determined that the data is not ECG data, the method proceeds to step 225 and performs an EEG data algorithm, an example of which is detailed in FIG. 3 and described herein. After the method performs the EEG data algorithm, the method proceeds to step 250. If at decision step 220, it is determined that the data is ECG data, the method proceeds to step 230 and forms R-R intervals. Step 230 is followed by step 235 At step 235, an accepted PD2i algorithm is run, an example of which is detailed in FIG. 4 and described herein. The method then proceeds to decision step 240 to determine if the PD2i values are ≦1.4. If the PD2i values are not ≦1.4, the method proceeds to step 275. If the PD2i values are ≦1.4, the method proceeds to step 245 and performs an outlier removal algorithm, an example of which is detailed in FIG. 5 and described herein.

After performing the outlier removal algorithm, the method proceeds to step 250 and runs the accepted PD2i algorithm. The method then proceeds to decision step 255. At decision step 255, it is determined if the PD2i values are ≦1.4. If the PD2i values are ≦1.4, the method proceeds to decision step 260. At decision step 260, it is determined if % N of accepted PD2i's is >30%. If the % N of accepted PD2i's is not >30%, the method proceeds to step 265 and is designated as rejected because of low % N. If, however, at decision step 260, the % N of accepted PD2i's is >30%, the method proceeds to step 270 and is designated as a positive PD2i test. The method then terminates.

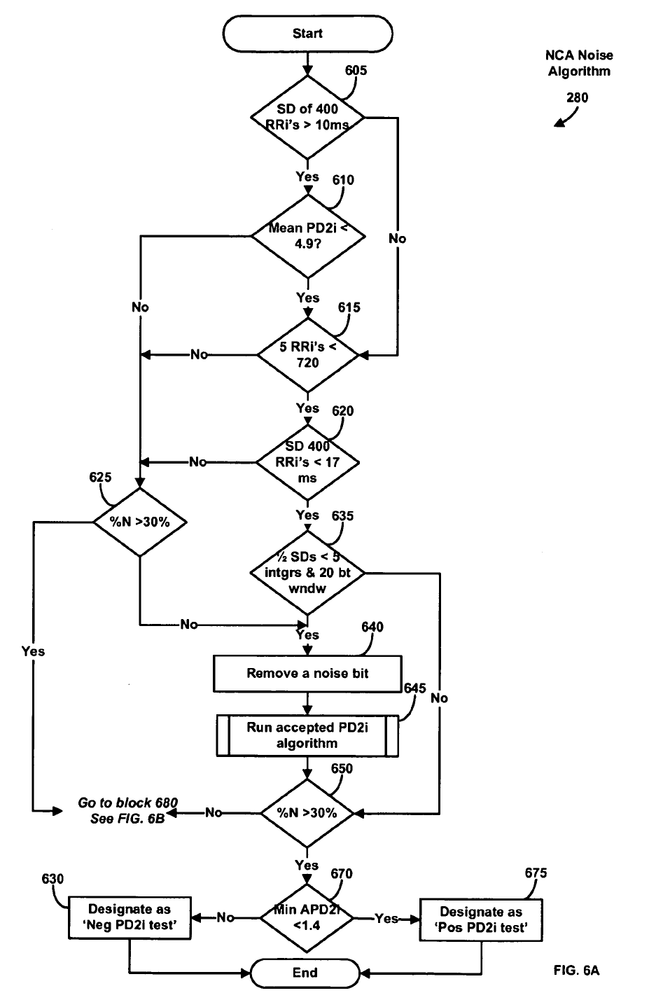

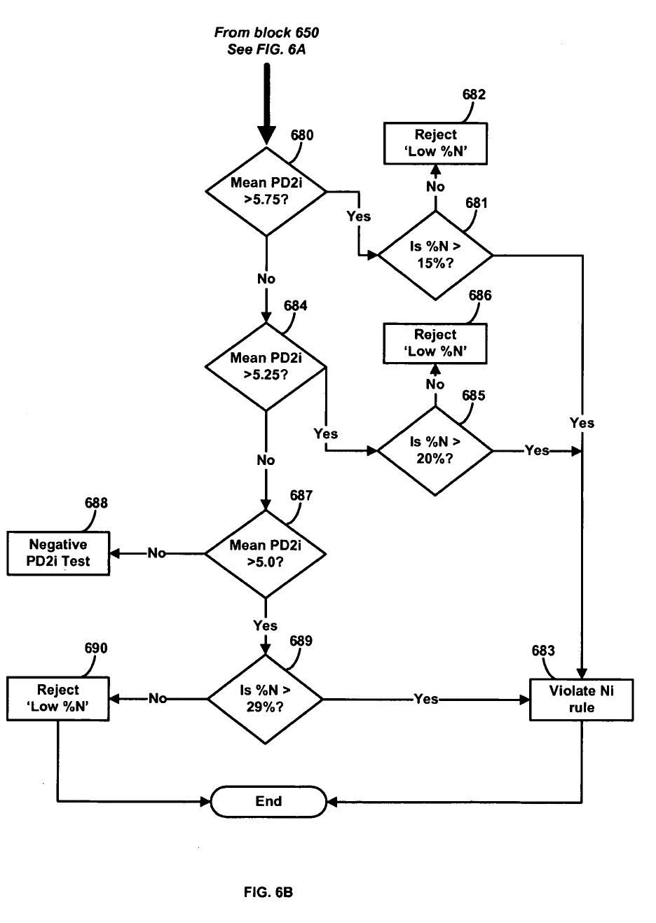

Turning back to decision step 255, if it is determined if the PD2i values are not ≦1.4, the method proceeds to decision step 275. At decision step 275, it is determined if the accepted PD2i values are >1.6. If the accepted PD2i values are >1.6, the method proceeds to step 280 and performs an NCA noise correction algorithm to determine if a designation of positive PD2i test, negative PD2i test, or rejected test as a result of low % N or Ni rule violation is warranted, an example of which is detailed in FIGS. 6 A and B and described herein. After performing the NCA noise correction algorithm, the method terminates.

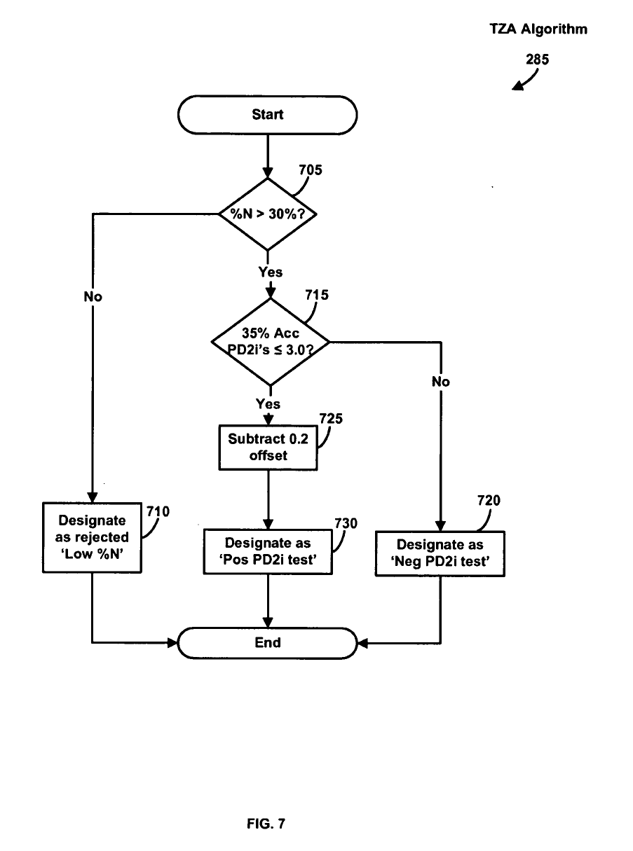

Turning back to decision step 275, if it is determined that the accepted PD2i values are not >1.6. The method proceeds to step 285, and performs a TZA noise correction algorithm to determine if a designation of positive PD2i test, negative PD2i test, or rejected test as a result of low % N is warranted, an example of which is detailed in FIG. 7 and described herein. After performing the TZA noise correction algorithm, the method terminates.

FIG. 3 illustrates an exemplary EEG data algorithm. The algorithm starts at step 305, where the data is filtered. Step 305 is followed by step 310. At step 310, linearity criteria are selected. Step 310 is followed by step 315. At step 315, a plot length is selected. Step 315 is followed by step 320. At step 320, a Tau is selected. Step 320 is followed by step 325. At step 325 convergence criterion are selected. Step 325 is followed by step 330. At step 330, an accepted PD2i algorithm is performed, an example of which is detailed in FIG. 4 and described herein. After performing step 330, the EEG data algorithm terminates.

Turning now to FIG. 4, this figure is a flow chart illustrating an exemplary PD2i subroutine 225, which begins at step 410. In step 410, PD2i subroutine 225 receives electrophysiological data. While this is shown as a separate step, this data corresponds to the indicator signals received from the subject. Step 410 is followed by step 415. In step 415, the PD2i subroutine 225 calculates the vector difference lengths. More specifically, the PD2i subroutine 225 calculates the vector difference lengths, finds their absolute values, and then rank orders them. A single vector difference length is made between a reference vector that stays fixed at a point i and any one of all other possible vectors, j, in the data series, with the exception of when i=j, in which case the value of zero is disregarded. Each vector is made by plotting, in a multidimensional space called an embedding dimension, m. The coordinates of this dimension are defined by the values of m, which are in actuality the number of successive data points, considering Tau, at each data point in the “Gamma” data series. That is, a short segment of the gamma-enriched data is used to form the coordinates to make an m-dimensional vector. For example, 3 data points make a 3-dimensional vector (m=3), 12 make a 12-dimensional vector (m=12). After calculating the reference vector, starting at a data-point i, and the j-vector (one of any other vectors that can be made), then the vector difference is calculated and its absolute value is stored in an array. All j-vectors are then made with respect to the single fixed i-vector. Then point-i is incremented and again all i-j vector difference lengths are again determined. Then m is incremented and the whole i-j vector difference lengths are again calculated. Essentially, these steps illustrate how the PD2i subroutine 225 completes step 420.

Step 420 is followed by step 425. In this step, the PD2i subroutine 225 calculates the correlation integrals for each embedding dimension (e.g., m point-i in the enriched gamma data series), where the fixed reference vector is located. These correlation integrals indicate generally the degrees of freedom at a particular point in time, depending upon the scaling interval. Step 425 is followed by step 430 where the PD2i subroutine 225 uses the correlation integral determined in step 425. Then this subroutine restricts the scaling region to the initial small-end of the correlation integral that lies above the unstable region caused by error resulting from the speed of the digitizer. More specifically, this subroutine defines a correlation integral scaling region based on the plot length criterion. This criterion essentially restricts the scaling to the small log-R end of the correlation integral with the property of insensitivity to data non-stationarity.

Step 430 is followed by the decision step 435. In this step, PD2i subroutine 225 determines whether the linearity criterion is satisfied. The linearity criterion makes the scaling region essentially linear and precludes it containing the floppy tail. If the linearity criterion is satisfied, the “yes” branch is followed from step 435 to step 440. In step 440, the PD2i subroutine 225 determines whether the minimum scaling criterion is satisfied, which essentially means that there are a suitable number of data points within the region. If the minimum scaling criterion is not satisfied, the PD2i subroutine 225 follows the “no” branch from step 435 to step 445. Step 445 also follows step 440 if the linearity criterion is not satisfied. In step 445, the PD2i subroutine 225 stores the mean, or average, slope and standard deviation as a −1.

When the minimum scaling criterion is satisfied, the “yes” branch is followed from step 440 to step 450. In step 450, the PD2i subroutine 225 stores the mean slope and deviation of the scaling region slopes of the correlation integrals for the convergent embedding dimensions. That is, the values are for the slopes where increasing m does not lead to a change in the slope of the scaling region for the associated point at a time i.

Step 455 follows step 445 and both steps 470 and 475. In step 455, the PD2i subroutine 225 selects the next PD2i point, which has either an i or an m increment.

Step 455 is followed by decision step 460. In this step, the PD2i subroutine 225 determines whether all the PD2i points and m s are selected. If there are remaining unselected values, the “no” branch is followed from step 460 to step 415, which essentially repeats the subroutine 225 iteratively until all i at each m have been calculated. If it is determined that all are selected at step 460, the PD2i subroutine 225 terminates.

Returning to decision step 465, the PD2i subroutine 225 determines whether the convergence criterion is satisfied. Essentially, this criterion analyzes the convergent PD2i slope values and determines if they converged more than a predetermined amount. If the convergence criterion is satisfied, step 465 is followed by step 470 (i.e., follow the “yes” branch). In this step, the PD2i subroutine 225 displays “Accepted.” If it is determined that the convergence criterion is not satisfied, the “no” branch is followed from step 465 to step 475 and branched to stem 445. In step 475, the PD2i subroutine 225 displays “Not Accepted.” In other words, “Not Accepted” indicates that the PD2i is invalid for some reason, such as noise, and stores the value −1 in step 445.

FIG. 5 illustrates an exemplary outlier removal algorithm. The algorithm starts at step 510, where the algorithm identifies a first R-R Interval outside a deviation threshold. This R-R Interval is an outlier. The deviation threshold can be, for example, 3 standard deviations. Step 510 is followed by step 515. At step 515, a linear spline for the outlier is defined. Step 515 is followed by step 520. At step 520, the outlier is overwritten with the spline. Step 520 is followed by step 525. At step 525, the algorithm increments to the next outlier. Step 525 is followed by decision step 530. At decision step 530 it is determined if the end of the file has been reached, that is, if i=Ni, where i is the current location in the file and Ni is the number of data points in the file. If it is determined that i≠Ni, the algorithm returns to step 510. If at step 525, it is determined that i=Ni, then algorithm terminates.

FIGS. 6 A and B illustrate an exemplary NCA noise correction algorithm. The algorithm starts at decision step 605, where it is determined whether the SD of 400 successive RRi's is >than 10 milliseconds. If it is determined that the SD of 400 successive RRi's is ≦10 milliseconds, the algorithm proceeds to decision step 615, described herein. If at decision step 605, it is determined that the SD of 400 successive RRi's is >than 10 milliseconds, the algorithm proceeds to decision step 610. At decision step 610, it is determined if the mean PD2i is below a usual normal mean of 5.0 to 6.0. The determination can be made if the mean PD2i is <4.9. If it is determined that the mean PD2i is ≧4.9, the algorithm proceeds to decision step 625, described herein.

If, however, at decision step 610 it is determined that the mean PD2i is <4.9, the algorithm proceeds to decision step 615. At decision step 615, it can be determined if the RRi's go to low values, indicating high heart rate, at least once in a 15-minute data sample. The determination can be made if 5 or more R-R Intervals<720 ms. If less than 5 RRi<720 msec, the algorithm proceeds to decision step 625, described herein. If, however, at decision step 615 it is determined that 5 or more RRi <720 ms, the algorithm proceeds to decision step 620. At decision step 620, it is determined if the R-R Interval data are somewhat “flat,” with little heart rate variability. The determination can be made if the SD of 400 successive RRi's, of at least one segment, is less than 17 ms. If the SD of 400 successive RRi's, of at least one segment, is not less than 17 ms, the algorithm proceeds to decision step 625. At decision step 625, it can be determined if % N of accepted PD2i's is >30%. If at decision step 625, it is determined that the % N of accepted PD2i's is >30%, the algorithm proceeds to decision step 680, detailed in FIG. 6B and described herein. If, however, at step 625 it is determined that the % N of accepted PD2i's is >30%, the algorithm proceeds to step 640.

Returning to decision step 620, if it is determined that the SD of 400 successive R-R Intervals, of at least one segment, is less than 17 ms, the algorithm proceeds to decision step 635. At decision step 635, it can be determined if there is a small amount of noise in the data. The determination can be made if more than 50% of the running windows of 20 RRi have an SD>±5. If more than 50% of the running windows of 20 RRi do not have an SD>±5, the algorithm proceeds to decision step 650, described herein. If, however, at step 635, it is determined that more than 50% of the running windows of 20 RRi have an SD>±5, the algorithm proceeds to step 640. At step 640 a noise bit can be removed.

Step 645 follows step 640. At step 645, an accepted PD2i algorithm can be run, an example of which is detailed in FIG. 4 and described above. Decision step 650 follows step 645. At decision step 650, it can be determined if the % N of accepted PD2i's is >30%.

If it is determined that the % N of accepted PD2i's is not >30%, the algorithm proceeds to decision step 680, detailed in FIG. 6B and described herein. If at decision step 625, it is determined that the % N of accepted PD2i's is >30%, the algorithm proceeds to decision step 670. At decision step 670 it can be determined if a minimum accepted PD2i is <1.4. If it is determined that the minimum accepted PD2i is <1.4 the algorithm proceeds to step 675 and designates a positive PD2i test. If, at decision step 670, it is determined that the minimum accepted PD2i is not <1.4 the algorithm proceeds to step 630 and designates a negative PD2i test.

Turning to decision step 680 in FIG. 6B, a determination can be made if a mean PD2i is >5.75. If it is determined that the mean PD2i is not >5.75, the algorithm proceeds to decision step 684, described herein. If, at decision step 680, it is determined that the mean PD2i is >5.75, the algorithm proceeds to decision step 681. At decision step 681, it can be determined if a % N of accepted PD2i's is >15%. If the % N of accepted PD2i's is not >15%, the algorithm proceeds to step 682 and rejects the test for low % N and ends. If, at decision step 681, the % N of accepted PD2i's is >15%, the algorithm proceeds to step 683 and declares a Ni rule violation. The algorithm then proceeds to designate a negative PD2i test at step 689. The algorithm terminates after step 689.

Returning to decision step 684, a determination can be made if a mean PD2i is >5.25. If it is determined that the mean PD2i is not >5.25, the algorithm proceeds to decision step 687, described herein. If, at decision step 684, it is determined that the mean PD2i is >5.25, the algorithm proceeds to decision step 685. At decision step 685, it can be determined if a % N of accepted PD2i's is >20%. If the % N of accepted PD2i's is not >20%, the algorithm proceeds to step 686 and rejects the test for low % N and ends. If, at decision step 685, the % N of accepted PD2i's is >20%, the algorithm proceeds to step 683 and declares an Ni rule violation. The algorithm terminates after step 683.

Returning to decision step 687, a determination can be made if a mean PD2i is >5.0. If it is determined that the mean PD2i is not >5.0, the algorithm proceeds to decision step 688, and declares a negative PD2i test and ends. If, at decision step 687, it is determined that the mean PD2i is >5.0, the algorithm proceeds to decision step 689. At decision step 689, it can be determined if a % N of accepted PD2i's is >29%. If the % N of accepted PD2i's is not >29%, the algorithm proceeds to step 690 and rejects the test for low % N. If, at decision step 689, the % N of accepted PD2i's is >29%, the algorithm proceeds to step 683 and declares an Ni rule violation. The algorithm terminates after step 683.

FIG. 7 illustrates an exemplary TZA noise correction algorithm. The TZA algorithm starts at decision step 705, where it can be determined if a % N of accepted PD2i's is >30%. If the % N of accepted PD2i's is not >30%, the algorithm proceeds to step 710 and designates the test as rejected for low % N and ends. If, at decision step 705, the % N of accepted PD2i's is >30%, the algorithm proceeds to decision step 715. At decision step 715, it can be determined if a percentage of accepted PD2i's are <3.0, the percentage can be, for example, 35, 45, 55, 65, 75, and the like. At decision step 715, it can be determined if >35% accepted PD2i's are <3.0. If >35% accepted PD2i's are not <3.0, the algorithm proceeds to step 720 and designates a negative PD2i test and ends. If, at decision step 715, >35% accepted PD2i's are ≦3.0, the algorithm proceeds to step 730 and designates a positive PD2i test and ends.

In another aspect, the automated software described in FIGS. 8A and 8B uses a computational method for determining a PD2i as the restricted scaling interval of the convergent slope of the correlation integral in conjunction with the various noise-handling algorithms and parameters, that are described herein.

FIG. 8A shows that first the ECG data are converted to R-R intervals (RRi) using a 3-point running window operator to identify successive R-wave peaks (one maxima). Then the accepted PD2i's are calculated. Accepted PD2i's are those PD2i values that meeting the Linearity Criterion, Convergence Criterion, and 10-point Minimum criteria, that occur within the Plot Length, become the Accepted PD2i's. The ratio of Accepted PD2i's to all PD2i's is calculated as % N. The Minimum PD2i of the accepted PD2i's is then found to lie in one of three intervals: a) >1.6, b) ≦1.6 and >1.4, or c) ≦1.4 (Select Range of PD2i's).

If the Minimum PD2i of the accepted PD2i's is in interval c, then RRi is inspected for outliers, and if outliers >3 Standard Deviations (SD) of mean RRi are found within a −12 to +12 data-point interval centered around the first PD2i</=1.4 (yes), then all outliers are removed by overwriting each with a linear interpolation spline from RRi of i−2 to i+2 centered on the detected outlier at point-i. A flag can be set so that if outliers have been removed, this routine will not run again. Minimum PD2i is then recalculated and retested for intervals a, b, c. If the Minimum PD2i remains in c, then it is examined for % N. If % N is >30% then positive PD2i is displayed. If outliers have been removed and recalculation of PD2i's has occurred, the file is rejected (reject PD2i Test) if it fails the % N is ≦30%.

FIG. 8B shows the TZA and NCA pathways that will be selected if the direct path described in FIG. 8A is not selected. If the NCA pathway is selected (interval a), outliers greater than 3 SD of the RRi are removed. A flag can be set so that this will not happen a second time. After the outliers are removed, the RRi is examined for four criteria of the NCA. If all are met (yes) then a noise-bit is removed from each RRi; a flag can be set so that this operation can only happen once. Then the PD2i's are again calculated and the accepted ones identified. If % N is >30%, then the PD2i's are again examined for the a, b, and c ranges and the range selected; if the range is c) (PD2i≦1.4), then the test is declared positive and the program exits. If the a) range (PD2i>1.6), then the test is declared negative and then exits. If the range is b) (PD2i ≦1.6 and >1.4), then the NCA test is shifted to the TZA test and the latch switch is moved to position #2 (*); the latch switch can be reset upon exiting.

In the TZA pathway, first the % of Accepted PD2i's less than 3.0 are found and if they are greater than 35% (yes) then 0.2 dimensions are subtracted from all of the PD2i's and the test is declared positive and exits. If the TZA criterion is not met (no) then the TZA is negative and the PD2i Test is declared negative through #2 of the latch switch and exits.

If the initial range selection is for a) (PD2i≦1.6 and >1.4), then the same % Accepted PD2i's less than 3.0 is examined, and if met (yes) then the test is positive. If the criterion is not met, then the test is transferred through the #1 position of the latch switch to the NCA, but then the latch switch is moved to position #2 to prevent a continuous loop and to declare the test negative if it happens again to come back from the NCA to the TZA test again because the Minimum PD2i is still in the transition zone; the latch switch can be reset to #1 upon exit.

### IV. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit scope. Efforts have been made to ensure accuracy with respect to numbers (e.g., amounts, thresholds, etc.), but some errors and deviations should be accounted for.

A. Comparison of Heartbeat PD2i Results by Hand Analysis vs Automated Analysis for a Large Database

Comparison of the blinded calculation of results by two different methods, using the same large number of patient files (340 ER patients, Pilot Data for SBIR, JE Skinner, PI, with outcomes known after calculation of first set of results), showed that 77% of results were the same. The two methods were Hand Analysis vs Automated Analysis of the identical, but blinded and coded, ECG files. All of the 21 Arrhythmic Death (AD) cases were the same for both methods (note 1 additional AD was found during the second set of calculations). For the remainder, the change in the results from the original to those using the automated software are shown in Table 2 below.

TABLE 2
Changes in Database Using Automation.

| Number of | Original - |
|---|---|
| ECG Files | Automated |
| 29 | % N-neg |
| 5 | % N-pos |
| 23 | pos-neg |
| 6 | neg-pos |
| 2 | PM-neg |
| 1 | PM-pos |

All files were from ER patients who lived at least 1-yr (i.e., non-AD patients).

The same noise handling algorithms (% N, NCA, TZA, removal of outliers, Ni-rule) were used in both sets of analyses. What is significant is that 29 files that were originally Rejected (% N) became True Negatives (i.e., patients lived for the 1-yr follow-up). Of equal significance, 23 original False Positives became True Negatives using the automation. One AD subject was rejected originally because the file was too short and therefore not added to the database, but with automation, it was noted that there were sufficient data according to the Ni-rule for a valid calculation at the lower PD2i values. The automation correctly changed 6 True Negative files to False Positives because of the more accurate calculations. Furthermore the automation detected 3 subjects, which were originally rejected because of having pacemakers (PM), that were actually found to have the pacemakers off; that is, the pacemakers were not providing demand pacing at the time ECG was recorded.

The explanation for the 29 changes of % N to True Negative in the database results is that the automated version recognized that the rejected files had high mean PD2i's and therefore violated the % N rule (% N<30) because of violation of the Ni-rule (Ni<10 exp PD2i); that is, the automated software applied both rules and showed the files to have sufficient data and thus an acceptable % N value. Five additional % N Reject files became Positives (False Positives). As they actually had % N>30. The explanation for the changes in the 23 False-Positive to True-Negative outcome is that better removal of outliers occurred during automation, which removed the Correlation Integral scaling for low PD2i's caused by the remaining outliers.

Automation of PD2i calculations results in more consistent application of the noise-handling algorithms (% N, NCA, TZA, removal of outliers, and Ni-rule) and thus reduces rejection-rates and false-positive rates for a large database of subjects.

B. PD2i of Heartbeats: Neural Regulation is the Final Link in the Mechanism Underlying Ventricular Fibrillation

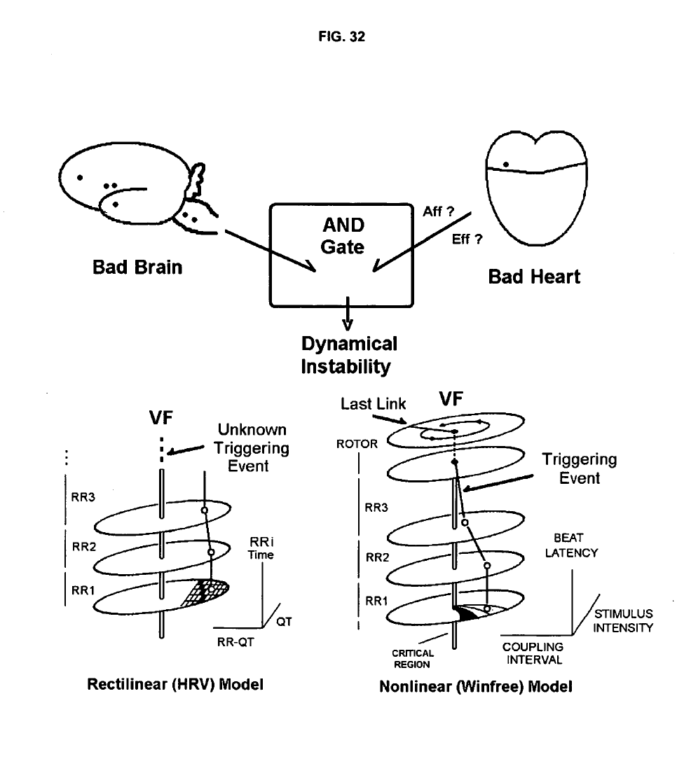

The text herein will refer to FIG. 32, which shows that both a “Bad Heart” and a “Bad Brain” are required to cause the dynamical instability of ventricular fibrillation (VF). For example, after cardiac denervation or cerebral blockade at specific sites (dots), coronary artery occlusion does not result in VF; usually, however, VF occurs in association with some kind of myocardial ischemia (see review by Skinner, 1987).

Whether it is the efferent input to the AND gate from the Bad Heart (Eff?) or its afferent input (Aff?), which loops through the cerebral centers (dots), is not yet known. It is noteworthy, however, that direct electrical stimulation of the cerebral centers (dots), can cause VF in a normal heart (see Skinner, 1985; 1987).

The Rectilinear (HRV) Model is based on the simple proposition that inotropy and chronotropy are the two variables that regulate the heartbeats. The QT interval is known to be an inverse measure of cardiac inotropy (contraction strength) and the RR-QT is known to be an inverse measure of cardiac chronotropy (heart rate). Thus the statement makes sense that each RRi interval has a QTi sub-epoch and an RRi-QTi sub-epoch, where in the model the sub-epochs are laid out in a rectilinear grid (checker board) and their sum is equal to RRi. That is, in FIG. 32 (left) the QT and RR-QT in a planar disks determines the RR length at which the next planar disk appears above it. This is simple arithmetic.

The conventional measures of heart rate variability (HRV) are based on the variability of RRi, which according to empirical results in animals (Skinner et al., 1991) and patients (retrospective, Skinner, Pratt, Vybiral, 1993; prospective, Skinner et al., 2005) is predictive of later ischemia-induced VF (arrhythmic death, AD). It does not matter whether QT and RR-QT define the rectilinear grid or 1/QT and 1/RR-QT define it, for each point in either two-dimensional plane will have an equivalent point in the other, and both are rectilinear.

The Rectilinear Model shows that inotropy and chronotropy are the two variables controlling RRi (i.e., is two dimensional), but it is quite similar to the Nonlinear (Winfree) Model (FIG. 32, right) with regard to its three axes. The Nonlinear Model, described by Winfree (1983, 1987), is a three dimensional model, because the time dimension (Beat Latency or RRi) “breaks down” and thus is another independent variable.

Winfree's model is based upon computer simulations of the nonlinear Goldman, Hodgkin, Huxley equations for the sodium, potassium and chloride membrane conductances in an excitable medium, and it is influenced by the experiments of Mines (1914), who first showed that the R-on-T injection of current into the excitable medium (isolated rabbit heart) would often lead to tachycardia and/or VF. Beat Latency (time) is not always completely determined by Stimulus Intensity and Coupling Interval, but usually it is. Winfree's three variables are: 1) injected stimulus intensity, 2) coupling interval, the time in the cardiac cycle at which the current is injected, and 3) latency (time) to the next beat. His computer simulation graphs revealed pie-shaped colors representing isochrones of latency that were plotted on the two dimensional plane of coupling interval and stimulus intensity.

FIG. 32 shows both a “Bad Brain” and a “Bad Heart” appear to be have an effect in determining the dynamical instability that leads to fatal ventricular fibrillation (VF) with either model. Rectilinear (left) and Nonlinear (right) Models of RRi generation (R1, R2, R3, . . . ) are shown. The Rectilinear (HRV) Model does not explain how VF is caused, but the Nonlinear (Winfree) Model does. In the latter, when the Beat Latency trajectory (connected dots) through the Stimulus Intensity and Coupling Interval plots (disks, similar to the QT vs RR-QT plots) lands on the critical region (point singularity and/or its immediate surround), it then mathematically (i.e., via the GHK equations for excitability) initiates a Rotor (rotating spiral wave). This initiation is like the R-on-T phenomenon, but current injection into the excitable medium at the same phase of the T-wave does not always initiate VF. There is one last link (Last Link) in which the refractoriness of the excitable medium is shortened by the nervous system to allow the rotor wave front to form.

In FIG. 32 (right) the injection of current in the pie-shaped isochrones, (i.e., colors) determined the latency in the next disk above it, except in the case where the isochrons came together and spiraled tightly around a critical point or “point singularity,” as he called it (critical region). Current injection in the point singularity, as in the Mines experiments, resulted in a rotating spiral wave (ROTOR) that looked very much like VF. That is, the model mathematically (i.e., by the nonlinear GHK equations) resulted in VF. Winfree called this mathematical spiral wave a “rotor,” as it was not a single rotating loop, but one filled in with concentric loops all having the same wavefront of depolarization (i.e., the radial line in the top disk). Winfree's interpretation was that Sudden Cardiac Death was a topological (mathematical) problem (Winfree, 1983).

Such mathematical rotors, with graded loops of circulation, have been observed in real physiological VF in a real myocardium (Gray, Pertsov and Jalife, 1998). Interestingly the outer loop of the rotor was earlier observed by Gordon Moe and associates in computer simulations using less powerful computers (Moe and Rheinboldt, 1964) that were motivated by physiological studies of VF initiation in which the refractory period of the myocardium was of major importance (Moe, Harris Wiggers, 1941).

The Rectilinear and Nonlinear Models at first glance appear to be quite similar. RR-QT is the same as the Coupling Interval. QT is a measure of how hard the heart contracts (actually 1/QT) and Stimulus Intensity, like QT, determines how hard the heart will contract. Latency to the next beat is also the same in both models. In the Rectilinear Model, RRi is the sum of QTi and RRi-QTi, and therefore not an independent variable (i.e., dimension or degree of freedom). In the Winfree Model the latency, expressed by isochrones (colors painted on the two dimensional disks) are pie-shaped, and thus are quite distinct from those rectilinear isochrones in the Rectilinear model (e.g., compare the dark-filled isochrones). The Nonlinear Model, however, has an isochron (critical point) that is potentially all colors in that all latencies are possible.

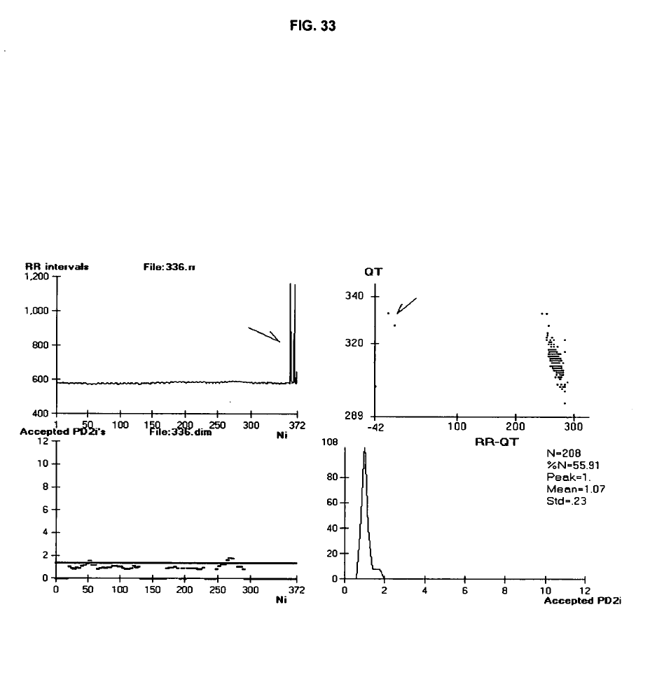

The Rectilinear Model does not match well to real physiological data. For example, the QTi vs RR-QTi should be a straight negative sloped line (Frank-Starling Law), but it is not (FIG. 33, upper right) and the “jitter” around it is not noise (i.e., because the PD2i of RRi is small, not infinite).

Although the Winfree model has a sound mathematical and physiological basis for both initiating and sustaining a rotor (Jalife and Berenfeld, 2004), when it comes to real physiological VF, however, things are a little more complex. The type of ischemia, heart size and species are also relevant (Rogers et al., 2003; Everett et al., 2005). But above all something of major importance has often been overlooked in most reviews—the role of the brain and nervous system in the causal mechanism of VF.

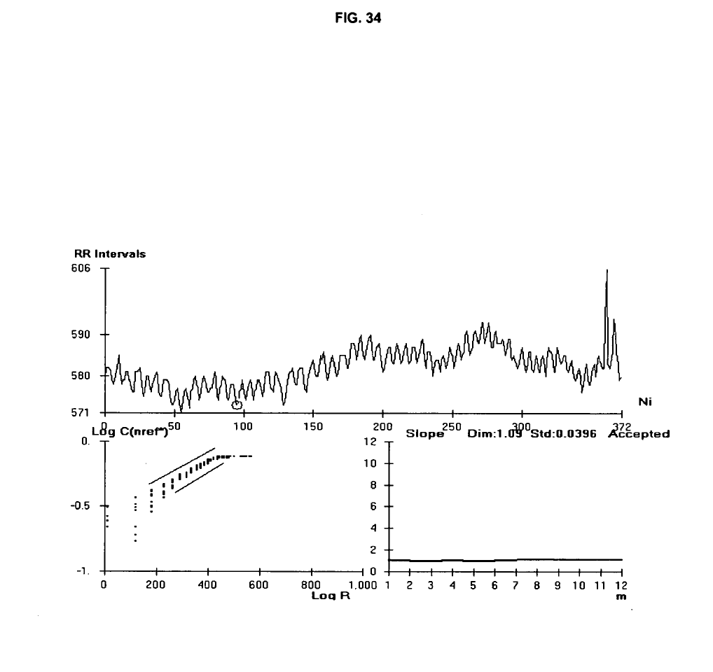

In FIG. 34-36 data is shown from a cardiac patient whose high-resolution ECG was recorded during the few minutes before VF. Although the RRi remained rather constant, the variation seen at higher gain (FIG. 34) showed 6 to 8 beat oscillations, which, being sinusoidal, naturally led to a mean PD2i around 1.00 (i.e., 1.07; all sinusoids have 1.00 degrees of freedom).

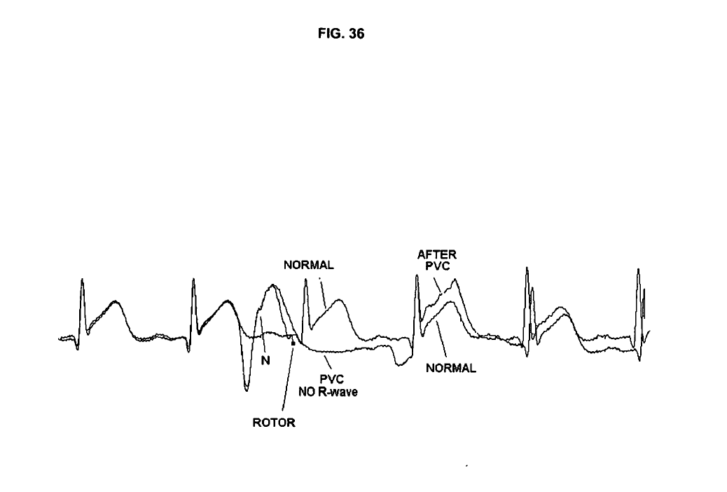

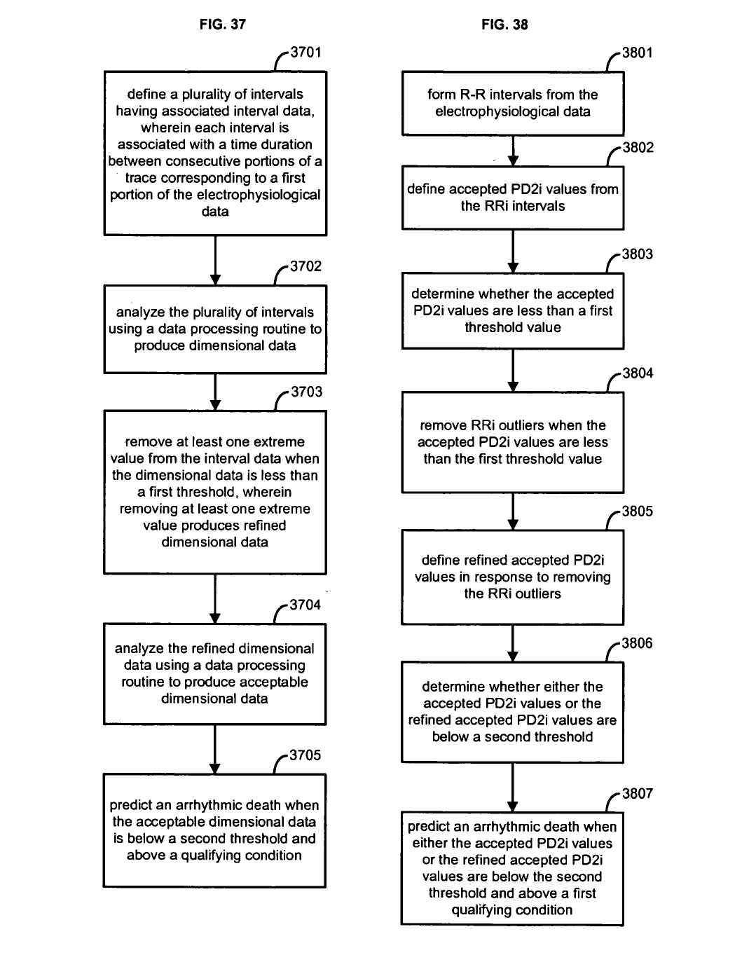

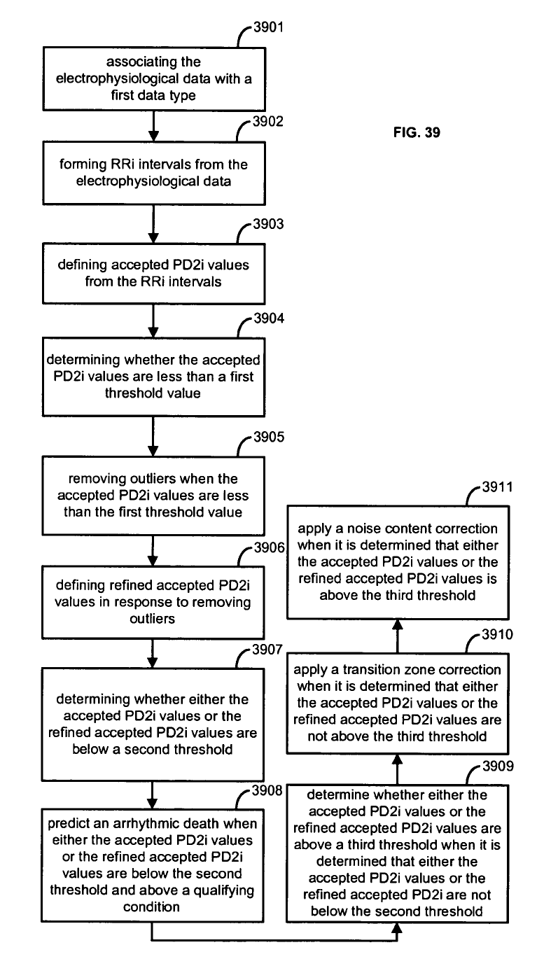

In the ECG of this AD patient, there were two ectopic premature ventricular complexes (PVCs), which are equivalent to current injections. Each PVC, as shown in FIG. 36, had identical amplitudes for their R-waves (deflection downward, as they were coming toward the electrode from a different direction) and identical coupling intervals that were precisely the same and completely overlapped. The two ectopic beats represented the same current injection at the same coupling interval, that is, as far as could be determined by the high-resolution ECG, yet one PVC resulted in VF and the other did not.

The difference observed for the two PVC's was that after current injection there was a more rapid recovery from refractoriness for the one that led to the VF. That is, the refractoriness allowed the current injection to result in a rotor. This difference in the recovery from refractoriness must be related to the neural regulation of the myocardium, as denervation by peripheral transsection or central neural blockade will prevent the occurrence of VF following coronary artery occlusion (Skinner, 1985; 1987).

In Winfree's model, the refractoriness of the excitable medium is completely controlled by the outward potassium conductance linked to the depolarization caused by the sodium conductance (i.e., refractoriness remains constant). In real cardiac tissue there are other conductances turned on during recovery from refractoriness and perhaps one for sustaining VF (Jalife and Berenfeld, 2004). But what about its control on a beat-to-beat basis?

It is the nerves projecting throughout the myocardium that can release chemicals almost instantaneously and change the membrane conductances, on a beat-to-beat basis. This type of regulation of VF seems to have been overlooked, perhaps because of the strong focus of work on the isolated myocardium. Direct measures of cardiac refractoriness in vivo, during rapidly changing brain states known to alter cardiac vulnerability to VF, attest to this important neural regulation (Skinner, 1983).

It is the shorter refractoriness that is the final link in the causal event that leads to VF. Reduced PD2i, which is a predictor of AD (VF) in a defined clinical cohort, is also a predictor of whether or not the neural regulation is likely to shorten refractoriness. Since the PD2i of the heartbeats is a measure of the neural regulation of the heart (Meyer et al., 1996), it is expected that it is associated with whether or not this rapid recovery of refractoriness will occur. The evidence seen in FIG. 33-36 shows that the final link in the causal mechanism of VF is the neural regulation that determines whether or not ischemia-induced, ectopic, current-injection at the critical point in the Winfree Model will result in a rotor.

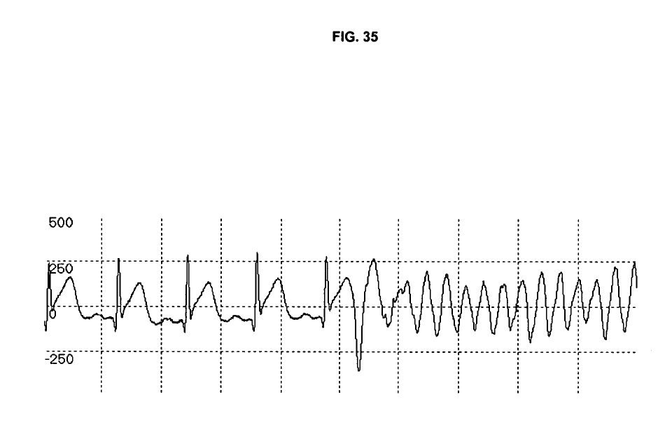

FIG. 33 shows a nonlinear analysis of the PD2i of the R-R intervals of an AD patient who showed two large PVCs (upper, arrows) one of which led to ventricular fibrillation (see FIGS. 35 and 36) and the other did not. The PD2i's of the last 28 points in the lower left quadrant were plotted from their Correlation Integrals, as they had only had 9 points in the Minimum Slope and were rejected by that criterion in the PD2i software; that is, the Minimum Slope criterion was changed from 10 to 9, which was thought to be legitimate because of the small Ni; the small Ni, however, was adequate by the Ni-rule, where Ni>10 exp PD2i.

FIG. 34 shows that the R-R intervals of the above AD patient are not really flat, but have a sinusoidal oscillation with a period of 6 to 8 heartbeats. The Correlation Integrals (M=1 to 12) at the lower left show linear scaling of about the same slope (slope=1) and rapid convergence in the plot of slope vs M, as seen at the lower right.

FIG. 35 shows that the ECG of the above AD patient in which a PVC (large downward deflection) occurs just after the peak of the last T-wave and initiates a small rapid rotor that then leads to a slower larger one. Note the ST-segment elevation indicative of acute myocardial ischemia (coronary insufficiency) is present.

FIG. 36 shows the coupling interval of the PVC that does not evoke a rotor (PVC No R-wave) and the one that does are precisely the same, as the downward deflections of both traces beginning at the far left overlap completely up to the T-wave peak. That is, the preceding R-R intervals at the left are identical and the notches (N) between the end of the ectopic R-waves of the two PVCs (ectopic R-deflection is downward) and the upward going T-waves are both completely overlapped. But the PVC that evokes the rotor shows a shorter recovery of the downward T-wave just before the beginning of the small amplitude rotor (ROTOR). The trace showing the remainder of the rotor has been ended (large dot) so as not to overwrite the other two traces; it can be seen completely in FIG. 35. This more rapid recovery from refractoriness appears to be the triggering event that allows the rotor to be initiated (i.e., not by the Winfree model). The reduced PD2i that predicts this susceptibility is due to the “cooperativity” among the heartbeat regulators (dots in FIG. 32). This indication of unique neural regulation of the heartbeats also appears to control the more rapid recovery from refractoriness, because neural blockade prevents VF in a pig model of coronary artery occlusion. The T-wave after the PVC that does not evoke the rotor shows suppression of the next R-wave (PVC, NO R-wave) and the occurrence of ripples in the next T-wave waveform (AFTER PVC); the latter that may indicate an aborted rotor that was stopped by the longer refractoriness. Post-current-injection control of refractoriness may be important in the mechanism of VF. The likelihood of having the short refractoriness appears to be inherent in the low-dimensionality of the heartbeat PD2i, as it accurately predicts the onset of VF.

In summary, the triggering event (FIG. 32) that leads mechanistically (i.e., mathematically) to VF in a model of an excitable medium, like the heart (FIG. 35), is not only related to its position in the Stimulus Intensity and Coupling Interval plane (i.e., color) in the Winfree Model, but it is also related to the neural control of refractoriness (FIG. 36) during the period immediately following its injection into the excitable medium. This neural mechanism is not addressed by the Winfree model, as it comes after the current injection, so the final link in the causal Triggering Event seen in FIG. 32 is the neural regulation that determines whether or not the RRi trajectory in the critical region is physiologically allowed to produce VF.

While the methods, systems, and computer readable media have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-expressed basis for interpretation, including: matters of logic with respect to arrangement of steps or operational flow; plain meaning derived from grammatical organization or punctuation; the number or type of embodiments described in the specification.

Throughout this application, various publications are referenced. The disclosures of these publications in their entireties are hereby incorporated by reference into this application in order to more fully describe the state of the art to which the methods, systems, and computer readable media pertain.

It will be apparent to those skilled in the art that various modifications and variations can be made without departing from the scope or spirit of the methods, systems, and computer readable media. Other embodiments will be apparent to those skilled in the art from consideration of the specification and practice of that disclosed herein. It is intended that the specification and examples be considered as exemplary only, with a true scope and spirit of the methods, systems, and computer readable media being indicated by the following claims.

What is claimed is:

1. An automated method of reducing noise associated with electrophysiological data for more effectively predicting an arrhythmic death, steps of the method comprising:
defining a plurality of intervals having associated interval data, wherein each interval is associated with a time duration between consecutive portions of a trace corresponding to a first portion of the electrophysiological data;
analyzing the plurality of intervals using a data processing routine to produce dimensional data;
removing at least one extreme value from the interval data when the dimensional data is less than a first threshold, wherein removing at least one extreme value produces refined dimensional data;
analyzing the refined dimensional data using a data processing routine to produce acceptable dimensional data; and
predicting an arrhythmic death when the acceptable dimensional data is below a second threshold and above a qualifying condition.

2. The method of claim 1, further comprising determining whether the electrophysiological data is either electroencephalogram data or electrocardiogram data.

3. The method of claim 2, further comprising a noise correction algorithm.

4. The method of claim 3, wherein the noise correction algorithm is selected from the group of noise correction algorithms consisting of an NCA noise correction algorithm and a TZA noise correction algorithm.

5. The method of claim 2, further comprising an EEG data algorithm when the electrophysiological data is electroencephalogram data.

6. The method of claim 5, wherein the EEG data algorithm further comprises the steps of:
selecting a linearity criterion;
selecting a plot length;
selecting a tau;
selecting a convergence criterion; and
defining the accepted PD2i values in response to selecting the linearity criterion, the plot length, the tau, and the convergence criterion.

7. The method of claim 1, wherein removing the at least one extreme value comprises the steps of:
identifying an outlying interval within the plurality of intervals, wherein the outlying interval is outside a deviation threshold;
defining a linear spline for the outlying interval; and
overwriting the outlying interval with the linear spline.

8. The method of claim 1, wherein the data processing routine is a PD2i algorithm.

9. The method of claim 1, wherein the first threshold is 1.4.

10. The method of claim 1, wherein the second threshold is 1.4.

11. The method of claim 1, wherein the qualifying condition is a percentage N of accepted or refined dimensional data is above a third threshold.

12. The method of claim 11, wherein the third threshold is 30 percent.

13. A method of reducing noise associated with electrophysiological data for more effectively predicting an arrhythmic death, steps of the method comprising:
forming RRi intervals from the electrophysiological data;
defining accepted PD2i values from the RRi intervals;
determining whether the accepted PD2i values are less than a first threshold value;
removing RRi outliers when the accepted PD2i values are less than the first threshold value;
defining refined accepted PD2i values in response to removing the RRi outliers;
determining whether either the accepted PD2i values or the refined accepted PD2i values are below a second threshold; and
predicting an arrhythmic death when either the accepted PD2i values or the refined accepted PD2i values are below the second threshold and above a first qualifying condition.

14. The method of claim 13, further comprising determining whether either the accepted PD2i values or the refined accepted PD2i values are above a third threshold when it is determined that either the accepted PD2i values or the refined accepted PD2i are not below the second threshold.

15. The method of claim 14, further comprising applying a transition zone correction when it is determined that either the accepted PD2i values or the refined accepted PD2i values are not above the third threshold.

16. The method of claim 15, wherein applying the transition zone correction further comprises the steps of:
determining whether either the accepted PD2i values or the refined accepted PD2i values are above the first qualifying condition;
determining whether a second qualifying condition for either the accepted PD2i values or the refined accepted PD2i values is less than a fourth threshold;
subtracting an offset from either the accepted PD2i values or the refined accepted PD2i values; and
predicting the arrhythmic death in response to subtracting the offset.

17. The method of claim 14 further comprising applying a noise content correction when it is determined that either the accepted PD2i values or the refined accepted PD2i values is above the third threshold.

18. The method of claim 13 further comprising classifying the electrophysiological data as electroencephalogram data.

19. The method of claim 13, wherein the first threshold is 1.4.

20. The method of claim 13, wherein the second threshold is 1.4.

21. The method of claim 13, wherein the first qualifying condition is a percentage N of accepted or refined dimensional data is above a fifth threshold.

22. The method of claim 21, wherein the fifth threshold is 30 percent.

23. The method of claim 14, wherein the third threshold is 1.6.

24. The method of claim 16, wherein the second qualifying condition is percentage of accepted or refined PD2i values less than 3.

25. The method of claim 16, wherein the fourth threshold is 35 percent.

26. A method of reducing noise associated with electrophysiological data for more effectively predicting an arrhythmic death, steps of the method comprising:
associating the electrophysiological data with a first data type;
forming RRi intervals from the electrophysiological data;
defining accepted PD2i values from the RRi intervals;
determining whether the accepted PD2i values are less than a first threshold value;
removing outliers when the accepted PD2i values are less than the first threshold value;
defining refined accepted PD2i values in response to removing outliers;
determining whether either the accepted PD2i values or the refined accepted PD2i values are below a second threshold;
predicting an arrhythmic death when either the accepted PD2i values or the refined accepted PD2i values are below the second threshold and above a qualifying condition;
determining whether either the accepted PD2i values or the refined accepted PD2i values are above a third threshold when it is determined that either the accepted PD2i values or the refined accepted PD2i are not below the second threshold;
applying a transition zone correction when it is determined that either the accepted PD2i values or the refined accepted PD2i values are not above the third threshold; and
applying a noise content correction when it is determined that either the accepted PD2i values or the refined accepted PD2i values is above the third threshold.

27. The method of claim 26 wherein applying a transition zone correction comprises:
subtracting an offset from either the accepted PD2i values or the refined accepted PD2i values; and
predicting the arrhythmic death in response to subtracting the offset.

28. The method of claim 26 wherein applying a noise content correction comprises:
removing an outlier greater than a predetermined number of standard deviations of the RRi intervals;
determining if the RRi intervals meet a predetermined number of NCA criteria;
removing a noise-bit from each RRi interval, if the predetermined number of NCA criteria are met;
re-defining accepted PD2i values from the RRi intervals; and
predicting the arrhythmic death in response to the redefined PD2i values.

29. The method of claim 26, wherein the first data type is selected from the group consisting of:
electroencephalogram data; and
electrocardiogram data.

30. The method of claim 26, wherein the first threshold is 1.4.

31. The method of claim 26, wherein the second threshold is 1.4.

32. The method of claim 26, wherein the third threshold is 1.6.

33. The method of claim 26, wherein the qualifying condition is a percentage N of accepted or refined dimensional data is above a fourth threshold.

34. The method of claim 33, wherein the fourth threshold is 30 percent.

35. A system for reducing noise associated with electrophysiological data used in predicting an arrhythmic death, comprising:
a processor coupled to receive the electrophysiological data;
a storage device with noise correction software in communication with the processor, wherein the noise correction software controls the operation of the processor and causes the processor to
form RRi intervals from the electrophysiological data;
define accepted PD2i values from the RRi intervals;
determine whether the accepted PD2i values are less than a first threshold value;
remove outliers when the accepted PD2i values are less than the first threshold value;
define refined accepted PD2i values in response to removing outliers;
determine whether either the accepted PD2i values or the refined accepted PD2i values are below a second threshold; and
predict an arrhythmic death when either the accepted PD2i values or the refined accepted PD2i values are below the second threshold and above a qualifying condition.

36. The system of claim 35, further comprising causing the processor to:
determine whether either the accepted PD2i values or the refined accepted PD2i values are above a third threshold when it is determined that either the accepted PD2i values or the refined accepted PD2i are not below the second threshold;
apply a transition zone correction when it is determined that either the accepted PD2i values or the refined accepted PD2i values are not above the third threshold; and
apply a noise content correction when it is determined that either the accepted PD2i values or the refined accepted PD2i values is above the third threshold.

37. The system of claim 36, further comprising causing the processor to:
subtract an offset from either the accepted PD2i values or the refined accepted PD2i values; and
predict the arrhythmic death in response to subtracting the offset.

38. The system of claim 36, further comprising causing the processor to:
remove an outlier greater than a predetermined number of standard deviations of the RRi intervals;
determine if the RRi intervals meet a predetermined number of NCA criteria;
remove a noise-bit from each RRi interval, if the predetermined number of NCA criteria are met;
re-define accepted PD2i values from the RRi intervals;
predict the arrhythmic death in response to the redefined PD2i values.

39. The system of claim 35, wherein the first data type is selected from the group consisting of:
electroencephalogram data; and
electrocardiogram data.

40. The system of claim 35, wherein the first threshold is 1.4.

41. The system of claim 35, wherein the second threshold is 1.4.

42. The system of claim 35, wherein the third threshold is 1.6.

43. The system of claim 35, wherein the qualifying condition is a percentage N of accepted or refined dimensional data is above a fourth threshold.

44. The system of claim 43, wherein the fourth threshold is 30 percent.

45. A computer readable medium having instructions to reduce noise associated with electrophysiological data for more effectively predicting an arrhythmic death, the instructions comprising the steps of:
forming RRi intervals from the electrophysiological data;
defining accepted PD2i values from the RRi intervals;
determining whether the accepted PD2i values are less than a first threshold value;
removing outliers when the accepted PD2i values are less than the first threshold value;
defining refined accepted PD2i values in response to removing outliers;
determining whether either the accepted PD2i values or the refined accepted PD2i values are below a second threshold; and
predicting an arrhythmic death when either the accepted PD2i values or the refined accepted PD2i values are below the second threshold and above a qualifying condition.

46. The computer readable medium of claim 45, further comprising instructions comprising the steps of:
determining whether either the accepted PD2i values or the refined accepted PD2i values are above a third threshold when it is determined that either the accepted PD2i values or the refined accepted PD2i are not below the second threshold;
applying a transition zone correction when it is determined that either the accepted PD2i values or the refined accepted PD2i values are not above the third threshold; and
applying a noise content correction when it is determined that either the accepted PD2i values or the refined accepted PD2i values is above the third threshold.

47. The computer readable medium of claim 46, further comprising instructions comprising the steps of
subtracting an offset from either the accepted PD2i values or the refined accepted PD2i values; and
predicting the arrhythmic death in response to subtracting the offset.

48. The computer readable medium of claim 46, further comprising instructions comprising the steps of:
removing an outlier greater than a predetermined number of standard deviations of the RRi intervals;
determining if the RRi intervals meet a predetermined number of NCA criteria;
removing a noise-bit from each RRi interval, if the predetermined number of NCA criteria are met;
re-defining accepted PD2i values from the RRi intervals;
predicting the arrhythmic death in response to the redefined PD2i values.

49. The computer readable medium of claim 45, wherein the first data type is selected from the group consisting of:
electroencephalogram data; and
electrocardiogram data.

50. The computer readable medium of claim 45, wherein the first threshold is 1.4.

51. The computer readable medium of claim 45, wherein the second threshold is 1.4.

52. The computer readable medium of claim 45, wherein the third threshold is 1.6.

53. The computer readable medium of claim 45, wherein the qualifying condition is a percentage N of accepted or refined dimensional data is above a fourth threshold.

54. The computer readable medium of claim 53, wherein the fourth threshold is 30 percent.
